(19) United States
(12) Patent Application Publication
(10) Pub. No.: US 2024/0293420 A1
(43) Pub. Date: Sep. 5, 2024

(54) PHARMACEUTICAL COMPOSITION, PREPARATION, AND PREPARATION METHOD THEREFOR AND USE THEREOF

(71) Applicant: WUHAN LL SCIENCE AND TECHNOLOGY DEVELOPMENT CO., LTD., Wuhan, Hubei (CN)

(72) Inventors: Huayun Hong, Wuhan, Hubei (CN); Jian Hang, Wuhan, Hubei (CN); Jun Lou, Wuhan, Hubei (CN); Li Liu, Wuhan, Hubei (CN); Wei Wu, Wuhan, Hubei (CN); Yongkai Chen, Wuhan, Hubei (CN); Yihan Zhang, Wuhan, Hubei (CN); Chaodong Wang, Wuhan, Hubei (CN)

(21) Appl. No.: 18/568,415

(22) PCT Filed: Jun. 10, 2022
(86) PCT No.: PCT/CN2022/098223

§ 371 Date: Dec. 8, 2023

(30) Foreign Application Priority Data
Jun. 10, 2021 (CN) 202110651136.9

(51) Int. Cl. A61K 31/5377; A61K 9/20; A61K 9/28

(52) U.S. Cl. CPC A61K 31/5377; A61K 9/2009; A61K 9/2013; A61K 9/2018; A61K 9/2054; A61K 9/2853; A61K 9/2866

(57) ABSTRACT

Disclosed in the present invention are a pharmaceutical composition, a preparation, and a preparation method therefor and the use thereof. The pharmaceutical composition contains an active ingredient and a pharmaceutically acceptable excipient. The active ingredient contains a compound represented by formula A. The compound represented by formula A is selected from one, two or more of the crystal form I, the crystal form III and the crystal form V. The excipient is selected-from or comprises, but is not limited to, one, two or more of the following excipients: a diluent, a disintegrant, an adhesive, a glidant and a lubricant. The pharmaceutical composition and the preparation of the present invention have a good safety and/or stability, and a high P2X3 antagonistic activity, and have less effect on the taste.

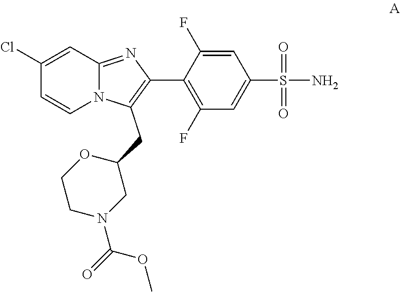 [SMILES: COC(=O)N1CCO[C@@H](Cc2c(-c3c(F)cc(S(N)(=O)=O)cc3F)nc3cc(Cl)ccn23)C1]

The present application claims the right of the priority of the prior application No. 202110651136.9 submitted to China National Intellectual Property Administration on Jun. 10, 2021, titled “PHARMACEUTICAL COMPOSITION, PREPARATION, AND PREPARATION METHOD THEREFOR AND USE THEREOF”. The contents of the prior application are incorporated herein by reference in its entirety.

### TECHNICAL FIELD

The present disclosure relates to the field of pharmaceutical compositions, and specifically relates to a pharmaceutical composition, a preparation, and a preparation method therefor and a use thereof.

### BACKGROUND

ATP receptors are classified into two main families, P2Y- and P2X-purinergic receptors, based on their molecular structure, transduction mechanism, and pharmacological properties. P2X-purinergic receptors are a family of ATP-gated cation channels and several subtypes have been cloned, including: six homomeric receptors, P2X1, P2X2, P2X3, P2X4, P2X5, and P2X7; and three heteromeric receptors, P2X2/3, P2X4/6, and P2X1/5. It has been found that P2X3 receptors are specifically expressed in the primary afferent nerve fibers of “hollow viscera”, such as lower urinary tract and respiratory tract.

Cough is the main symptom of respiratory diseases, and 70% to 80% of patients in respiratory clinics have cough symptoms. With the increasing prevalence of COPD, IPF, etc., and cough as the main symptom of most respiratory diseases, the demand is also increasing. As a defensive nerve reflex of the body, cough is beneficial to clear respiratory secretions and harmful factors, but frequent and severe cough will seriously affect the work, life, and social activities of patients.

There are not many varieties of P2X3 antagonists specifically developed for cough indications, and the current project with rapid progress is Roche's AF-219 project, which shows good efficacy on refractory cough in the latest completed phase II clinical trial, but with serious dysgeusia problem.

At present, there is no drug on the market that inhibits P2X3 pathway for the treatment of many conditions including chronic cough. Therefore, the development of new drugs that can inhibit the activity of P2X3 is of positive significance for the treatment of diseases.

### CONTENT OF THE PRESENT INVENTION

The present disclosure provides a pharmaceutical composition, comprising an active ingredient and a pharmaceutically acceptable excipient; the active ingredient comprises a compound of formula A:

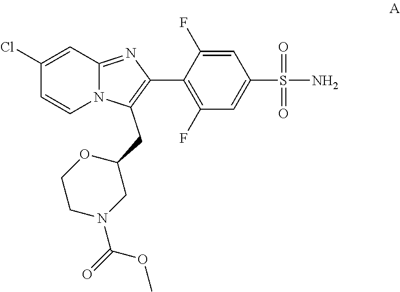 [SMILES: COC(=O)N1CCO[C@@H](Cc2c(-c3c(F)cc(S(N)(=O)=O)cc3F)nc3cc(Cl)ccn23)C1]

the excipient is selected from one, two or more of the following excipients including, but not limited to, a diluent, a disintegrant, an adhesive, a glidant, and a lubricant.

According to technical solutions of the present disclosure, the compound of formula A is selected from one, two or more of a crystal form I, a crystal form III, and a crystal form V.

According to technical solutions of the present disclosure, the crystal form I has characteristic peaks at 8.56°±0.20°, 12.48°±0.20°, and 22.13°±0.20° in an X-ray powder diffraction expressed at a 2θ angle using Cu-Kα radiation.

Further, the crystal form I has characteristic peaks at 8.56°±0.20°, 12.48°±0.20°, 22.13°±0.20°, 13.53°±0.20°, 14.25°±0.20°, 25.18°±0.20°, and 26.07°±0.20° in an X-ray powder diffraction expressed at a 2θ angle using Cu-Kα radiation.

Further, the crystal form I has characteristic peaks at 8.56°±0.20°, 12.48°±0.20°, 22.13°±0.20°, 13.53°±0.20°, 14.25°±0.20°, 25.18°±0.20°, 26.07°±0.20°, 22.32°±0.20°, 23.23°±0.20°, and 23.42°±0.20° in an X-ray powder diffraction expressed at a 2θ angle using Cu-Kα radiation.

Preferably, the crystal form I has an XRPD pattern substantially as shown in FIG. 3.

Preferably, the crystal form I has a differential scanning calorimetry pattern with an endothermic peak at about 152° C. and a melting enthalpy of about 44±2 J/g.

Preferably, the crystal form I has a thermogravimetric analysis pattern and a differential scanning calorimetry pattern substantially as shown in FIG. 4.

Preferably, the crystal form I has a polarizing microscope pattern substantially as shown in FIG. 5.

According to technical solutions of the present disclosure, the crystal form III has characteristic peaks at 12.91°±0.20°, 16.77°±0.20°, 19.27°±0.20°, and 22.80°±0.20° in an X-ray powder diffraction expressed at a 2θ angle using Cu-Kα radiation.

Further, the crystal form III has characteristic peaks at 12.91°±0.20°, 16.77°±0.20°, 19.27°±0.20°, 22.80°±0.20°, 13.75°±0.20°, 14.46°±0.20°, and 20.86°±0.20° in an X-ray powder diffraction expressed at a 2θ angle using Cu-Kα radiation.

Further, the crystal form III has characteristic peaks at 12.91°±0.20°, 16.77°±0.20°, 19.27°±0.20°, 22.80°±0.20°, 13.75°±0.20°, 14.46°±0.20°, 20.86°±0.20°, 21.08°±0.20°, 23.75°±0.20°, and 24.05°±0.20° in an X-ray powder diffraction expressed at a 2θ angle using Cu-Kα radiation.

Preferably, the crystal form III has an XRPD pattern substantially as shown in FIG. 10.

Preferably, the crystal form III contains 0.4 equivalents of water.

Preferably, the crystal form III has a thermogravimetric analysis pattern with a weight loss gradient of about 1.5% in the range of room temperature to 100° C.

Preferably, the crystal form III has a differential scanning calorimetry pattern with a first endothermic peak showing removal of 0.4 water.

Preferably, the thermogravimetric analysis pattern and differential scanning calorimetry pattern of the crystal form III are substantially as shown in FIG. 11.

Preferably, the crystal form III has a polarizing microscope pattern substantially as shown in FIG. 12.

According to technical solutions of the present disclosure, the crystal form V has characteristic peaks at 8.38°±0.20°, 9.15°±0.20°, 13.52°±0.20°, and 18.44°±0.20° in an X-ray powder diffraction expressed at a 2θ angle using Cu-Kα radiation.

Further, the crystal form V has characteristic peaks at 8.38°±0.20°, 9.15°±0.20°, 13.52°±0.20°, 18.44°±0.20°, 16.26°±0.20°, 16.89°±0.20°, and 17.86°±0.20° in an X-ray powder diffraction expressed at a 2θ angle using Cu-Kα radiation.

Further, the crystal form V has characteristic peaks at 8.38°±0.20°, 9.15°±0.20°, 13.52°±0.20°, 18.44°±0.20°, 16.26°±0.20°, 16.89°±0.20°, 17.86°±0.20°, 22.35°±0.20°, 23.56°±0.20°, and 24.74°±0.20° in an X-ray powder diffraction expressed at a 2θ angle using Cu-Kα radiation.

Preferably, the crystal form V has an XRPD pattern substantially as shown in FIG. 16.

Preferably, the crystal form V has a differential scanning calorimetry pattern with an endothermic peak at about 166° C. and a melting enthalpy of about 70±2 J/g.

Preferably, the crystal form V has a thermogravimetric analysis pattern and a differential scanning calorimetry pattern substantially as shown in FIG. 17.

Preferably, the crystal form V has a polarizing microscope pattern substantially as shown in FIG. 18.

According to technical solutions of the present disclosure, the compound of formula A has a particle size of 1 to 40 μm, for example 1.5 to 35 μm.

According to technical solutions of the present disclosure, the compound of formula A has a D10 particle size of 1 to 5 μm, for example 1.5 to 4 μm, exemplarily 2 μm, 2.21 μm, 2.5 μm, and 3 μm.

Further, the compound of formula A has a D50 particle size of 6 to 15 μm, for example 8 to 12 μm, exemplarily 9 μm, 10.2 μm, 11 μm, and 11.5 μm.

Further, the compound of formula A has a D90 particle size of 20 to 40 μm, for example 25 to 35 μm, exemplarily 27 μm, 29.1 μm, and 30 μm.

According to technical solutions of the present disclosure, the compound of formula A has a loose density (bulk density) of 0.2 to 0.3 g/mL, for example 0.22 to 0.28 g/mL, exemplarily 0.23 g/mL, 0.24 g/mL, 0.25 g/mL, 0.26 g/mL, 0.27 g/mL, and 0.29 g/mL.

According to technical solutions of the present disclosure, the compound of formula A has a tap density (bulk density) of 0.32 to 0.5 g/mL, for example 0.35 to 0.45 g/mL, exemplarily 0.33 g/mL, 0.36 g/mL, 0.38 g/mL, 0.40 g/mL, 0.42 g/mL, 0.44 g/mL, and 0.48 g/mL.

The pharmaceutically acceptable excipient is preferably an excipient which is chemically non-reactive with or inert to the active ingredient.

According to technical solutions of the present disclosure, the diluent can be selected from one, two or more of the following substances: lactose, microcrystalline cellulose, sucrose, glucose, mannitol, sorbitol, calcium sulfate, calcium gluconate, calcium hydrogen phosphate, calcium phosphate, calcium carbonate, calcium bicarbonate, starch, carboxymethyl starch, pregelatinized starch, etc., for example lactose and/or microcrystalline cellulose; and further, the lactose is lactose monohydrate.

For example, the diluent comprises a first diluent and a second diluent. The first diluent and the second diluent are different, and independently selected from one of the above diluents. Preferably, the first diluent is microcrystalline cellulose, and the second diluent is lactose monohydrate.

According to technical solutions of the present disclosure, the disintegrant can be selected from one, two or more of the following substances: croscarmellose sodium, pregelatinized starch, microcrystalline cellulose, alginic acid, lignocellulose, sodium carboxymethyl starch, guar gum, cross-linked polyvinylpyrrolidone, etc., for example croscarmellose sodium.

According to technical solutions of the present disclosure, the adhesive can be selected from one, two or more of the following substances: hydroxypropyl cellulose, gelatin, dextrin, maltodextrin, sucrose, gum arabic, polyvinylpyrrolidone, methyl cellulose, carboxymethyl cellulose, ethyl cellulose, polyvinyl alcohol, polyethylene glycol, and hydroxypropyl methyl cellulose, etc., for example hydroxypropyl cellulose; and further, the adhesive can be low-substituted hydroxypropyl cellulose or high-substituted hydroxypropyl cellulose.

According to technical solutions of the present disclosure, the glidant can be selected from one, two or more of the following substances: colloidal silica, silica, talc, calcium silicate, magnesium silicate, and calcium hydrogen phosphate, etc., for example colloidal silica.

According to technical solutions of the present disclosure, the lubricant can be selected from one, two or more of the following substances: magnesium stearate, calcium stearate, zinc stearate, talc, glycerol monostearate, polyethylene glycol (e.g. polyethylene glycol 4000, polyethylene glycol 6000, polyethylene glycol 8000), sodium benzoate, adipic acid, fumaric acid, boric acid, sodium chloride, sodium oleate, triacetin, polyoxyethylene monostearate, sucrose monolaurate, sodium chloride, sodium lauryl sulfate, and magnesium lauryl sulfate, etc., for example magnesium stearate, calcium stearate, and/or zinc stearate.

According to technical solutions of the present disclosure, the excipient may further comprise a corrigent. For example, the corrigent can be selected from one, two or more of the following substances: stevia, fructose, glucose, fructose syrup, honey, aspartame, protein sugar, xylitol, mannitol, lactose, sorbitol, flavor, and maltitol, etc.

According to one embodiment of the present disclosure, the diluent is lactose monohydrate and microcrystalline cellulose, the disintegrant is croscarmellose sodium, the adhesive is hydroxypropyl cellulose, the glidant is colloidal silica, and the lubricant is magnesium stearate.

According to technical solutions of the present disclosure, the pharmaceutical composition comprises the following components: the compound of formula A, lactose monohydrate, microcrystalline cellulose, croscarmellose sodium, hydroxypropyl cellulose, colloidal silica, and magnesium stearate.

In the pharmaceutical composition described herein, the content of each component can be selected as desired, for example, the content of the compound of formula A can be present in the pharmaceutical composition in a therapeutically effective amount.

According to technical solutions of the present disclosure, the pharmaceutical composition comprises 10 to 40 parts, for example 15 to 35 parts, exemplarily 12 parts, 18 parts, 20 parts, 23 parts, 25 parts, 27 parts, 30 parts, 32 parts, and 38 parts by weight of the compound of formula A.

According to technical solutions of the present disclosure, the pharmaceutical composition comprises 50 to 80 parts, for example 55 to 75 parts, exemplarily 52 parts, 58 parts, 60 parts, 63 parts, 65 parts, 70 parts, 72 parts, and 78 parts by weight of the diluent. Further, the first diluent and the second diluent have a weight ratio of (10 to 25):(40 to 55), for example (15 to 20):(40 to 55), exemplarily 17.4:49.6.

According to technical solutions of the present disclosure, the pharmaceutical composition comprises 0.5 to 6 parts, for example 1 to 5 parts, exemplarily 0.75 parts, 1.5 parts, 2 parts, 2.5 parts, 3 parts, 3.5 parts, 4 parts, and 5.5 parts by weight of the disintegrant.

According to technical solutions of the present disclosure, the pharmaceutical composition comprises 0.5 to 6 parts, for example 1 to 5 parts, exemplarily 0.75 parts, 1.5 parts, 2 parts, 2.5 parts, 3 parts, 3.5 parts, 4 parts, and 5.5 parts by weight of the adhesive.

According to technical solutions of the present disclosure, the pharmaceutical composition comprises 0.1 to 3 parts, for example 0.3 to 2 parts, exemplarily 0.5 parts, 1 part, 1.2 parts, 1.5 parts, and 2.5 parts by weight of the glidant.

According to technical solutions of the present disclosure, the pharmaceutical composition comprises 0.1 to 3 parts, for example 0.3 to 2 parts, exemplarily 0.5 parts, 1 part, 1.2 parts, 1.5 parts, and 2.5 parts by weight of the lubricant.

The sum of parts by weight of each component in the pharmaceutical composition is 100 parts.

According to technical solutions of the present disclosure, the pharmaceutical composition comprises the following components by weight: 10 to 40 parts of compound of formula A, a total of 50 to 80 parts of lactose monohydrate and microcrystalline cellulose, 0.5 to 6 parts of croscarmellose sodium, 0.5 to 6 parts of hydroxypropyl cellulose, 0.1 to 3 parts of colloidal silica, and 0.1 to 3 parts of magnesium stearate;

According to exemplary embodiments of the present disclosure, the pharmaceutical composition comprises the following components by weight: 25 mg of compound of formula A, 49.6 mg of lactose monohydrate, 17.4 mg of microcrystalline cellulose, 3.0 mg of croscarmellose sodium, 3.0 mg of hydroxypropyl cellulose, 1.0 mg of colloidal silica, and 1.0 mg of magnesium stearate;

According to exemplary embodiments of the present disclosure, the pharmaceutical composition comprises the following components by weight: 100 mg of compound of formula A, 198.4 mg of lactose monohydrate, 69.6 mg of microcrystalline cellulose, 12.0 mg of croscarmellose sodium, 12.0 mg of hydroxypropyl cellulose, 4.0 mg of colloidal silica, and 4.0 mg of magnesium stearate.

According to embodiments of the present disclosure, the pharmaceutical composition is in a solid form, such as a powdered solid form.

According to embodiments of the present disclosure, the pharmaceutical composition may be prepared in a dosage form suitable for administration.

The present disclosure also provides a pharmaceutical preparation, such as a solid preparation, comprising the pharmaceutical composition. Exemplarily, the solid preparation may be a tablet, a capsule, or a granule.

According to exemplary embodiments of the present disclosure, the tablet is a coated tablet, comprising a tablet core and a coating layer. Preferably, the tablet core comprises the pharmaceutical composition.

According to exemplary embodiments of the present disclosure, there is no particular limitation on the composition of the coating layer, which may be prepared, for example, by using a commercially available known gastric-soluble film coating premix, or according to known methods. For example, the coating layer further comprises one, two or more selected from polyvinyl alcohol, polyethylene glycol, hydroxypropyl methyl cellulose, hydroxypropyl cellulose, acrylic resin VI, polyvinylpyrrolidone, propylene glycol, castor oil, silicone oil, triglyceride, talc, titanium dioxide, and coloring agent. Film coating materials may also be purchased commercially, such as Opadry gastric-soluble coating series and “EASPRAY™” gastric-soluble coating series.

According to one technical solution of the present disclosure, the tablet core of the coated tablet comprises the following components by weight: 10 to 40 parts of compound of formula A, a total of 50 to 80 parts of lactose monohydrate and microcrystalline cellulose, 0.5 to 6 parts of croscarmellose sodium, 0.5 to 6 parts of hydroxypropyl cellulose, 0.1 to 3 parts of colloidal silica, and 0.1 to 3 parts of magnesium stearate;
the coating layer has a film coating material of Opadry gastric-soluble coating series;
preferably, the compound of formula A is present in the tablet core in the form of crystal forms I, III, and V.

Exemplarily, the tablet core of the coated tablet comprises the following components: 25 mg of compound of formula A, 49.6 mg of lactose monohydrate, 17.4 mg of microcrystalline cellulose, 3.0 mg of croscarmellose sodium, 3.0 mg of hydroxypropyl cellulose, 1.0 mg of colloidal silica, and 1.0 mg of magnesium stearate;
preferably, the compound of formula A is present in the tablet core in the form of crystal forms I, III, and V;
the coating layer has a film coating material of Opadry gastric-soluble coating series.

Exemplarily, the tablet core of the coated tablet comprises the following components: 100 mg of compound of formula A, 198.4 mg of lactose monohydrate, 69.6 mg of microcrystalline cellulose, 12.0 mg of croscarmellose sodium, 12.0 mg of hydroxypropyl cellulose, 4.0 mg of colloidal silica, and 4.0 mg of magnesium stearate;
preferably, the compound of formula A is present in the tablet core in the form of crystal forms I, III, and V;
the coating layer has a film coating material of Opadry gastric-soluble coating series.

The present disclosure further provides a method for preparing the pharmaceutical composition, comprising mixing the components comprised therein. Preferably, a prescribed amount of the compound of formula A, the glidant and the first diluent are first sieved (e.g., through a 60 mesh sieve), and then mixed with other components.

The present disclosure also provides a method for preparing the tablet, comprising compressing the pharmaceutical composition into a tablet, for example by a wet granulation tabletting method, and optionally with or without coating.

Preferably, the wet granulation tabletting method comprises: wet granulating the pharmaceutical composition except the lubricant, pelletizing, drying, and pelletizing again to obtain a dry granule; mixing the dry granule with the lubricant, and tabletting.

According to embodiments of the present disclosure, in the drying step of the wet granulation tabletting method, the weight loss on drying of the material is controlled at 1.5% to 2.5%. Preferably, in the drying step, when the weight loss on drying of the material is between 1.0% and 2.5%, an inlet air temperature is turned off to stop drying. Preferably, in the drying step, the equipment used is a fluidized bed.

According to embodiments of the present disclosure, in the wet granulating step, the stirring speed is 200 to 400 rpm, preferably 250.0 rpm; the cutting speed is 1000.00 to 2000.00 rpm, preferably 1500.0 rpm; the granulation time is 30 seconds to 2 minutes, preferably 60 seconds.

Further, the wet granulating comprises: mixing the compound of formula A, the glidant (e.g., colloidal silica), the diluent (e.g., microcrystalline cellulose and lactose monohydrate), and the disintegrant (e.g., croscarmellose sodium), spraying an adhesive solution (e.g., hydroxypropyl cellulose) into the mixture, and granulating after the adhesive solution is sprayed, optionally with or without water replenishing;
preferably, the adhesive solution is an aqueous solution of an adhesive, and further its concentration is 5 to 15%, for example 9%.

Preferably, the preparation of the mixture comprises:
mixing and sieving the compound of formula A, the glidant (e.g., colloidal silica), and the first diluent (e.g., microcrystalline cellulose) to obtain a mixture 1;
mixing and sieving the second diluent (e.g., lactose monohydrate) and the disintegrant (e.g., croscarmellose sodium) to obtain a mixture 2;
and mixing the mixture 1 and the mixture 2 to obtain a mixture. Preferably, a method for preparing a coated tablet comprises the following steps:
(1) wet granulating the mixture, pelletizing, drying, and pelletizing again to obtain a dry granule;
(2) mixing the dry granule with the lubricant (e.g., magnesium stearate), and tabletting to obtain a tablet core;
(3) spraying the tablet core with a coating solution to obtain the coated tablet.

The present disclosure also provides a method for storing the pharmaceutical composition or the preparation, comprising storing the pharmaceutical composition or the preparation in the dark. Further, storage conditions further comprise dry storage.

The present disclosure also provides a use of the pharmaceutical composition in the manufacture of a P2X3 inhibitor.

In the use, the P2X3 inhibitor can be used in mammalian organisms; it can also be used in vitro, mainly for experimental purposes, for example: as a standard sample or control sample to provide a comparison, or prepared as a kit according to conventional methods in the art to provide rapid detection of the inhibitory effect of P2X3.

The present disclosure also provides a use of the pharmaceutical composition in the manufacture of a pharmaceutical preparation, such as a solid preparation, further such as tablets, especially in the manufacture of a tablet by a direct tabletting method.

According to technical solutions of the present disclosure, the pharmaceutical preparation is a drug for preventing, curing, treating, or alleviating a disease in animals that is at least partially mediated by P2X3 or related to P2X3 activity; or, the drug is used for the treatment of pain, pruritus, endometriosis, urinary tract disease, or respiratory disease.

In some embodiments, the disease comprises pain; and the pain includes, but is not limited to: inflammatory pain, surgical pain, visceral pain, dental pain, premenstrual pain, central pain, pain due to burns, migraine, or cluster headache.

In some embodiments, the disease comprises a urinary tract disease; the urinary tract disease includes urinary incontinence, overactive bladder, dysuria, cystitis, prostatitis, prostatodynia, and benign prostatic hyperplasia;
the urinary incontinence comprises uncontrolled urinary incontinence, and the uncontrolled urinary incontinence is associated with urge urinary incontinence, cough urinary incontinence, stress urinary incontinence, overflow urinary incontinence, functional urinary incontinence, neurogenic urinary incontinence, post-prostatectomy urinary incontinence, urinary urgency, nocturia, and enuresis.

In some embodiments, the disease comprises a respiratory disease including, but not limited to, a respiratory disorder including idiopathic pulmonary fibrosis, chronic obstructive pulmonary disease, asthma, bronchospasm, acute cough, or chronic cough.

The chronic cough is a cough that lasts for more than eight weeks and causes serious adverse social, psychological, and physical effects.

In some embodiments, the disease comprises a respiratory disease including acute cough or chronic cough. The cough is an acute cough or a chronic cough, and the cough is associated with a disease, disorder, or condition selected from chronic obstructive pulmonary disease, asthma, tuberculosis, bronchitis, bronchiectasis, suppurative lung disease, respiratory malignancy, hypersensitivity, cystic fibrosis, pulmonary fibrosis, respiratory inflammation, emphysema, pneumonia, lung cancer, neoplasia of the lung, sore throat, common cold, influenza, respiratory infection, bronchoconstriction, sarcoidosis, viral or bacterial infection of the upper airway, angiotensin converting enzyme (ACE) inhibitor therapy, smoker's cough, chronic dry cough without phlegm, neoplastic cough, cough due to gastroesophageal reflux, and inhalation of irritants, smoke, fog, dust, or air pollution.

In some embodiments, the pruritus is associated with an inflammatory skin disease, an infectious skin disease, an autoimmune skin disease, or a pregnancy-related skin disease.

In some embodiments, the pruritus is associated with an inflammatory skin disease selected from atopic dermatitis, allergic contact dermatitis, irritant contact dermatitis, xerotic dermatitis, dysplastic dermatitis, lichen planus, lichen sclerosus, polymorphic eruption psoriasis, Graves disease, mucoid degeneration, mastocytosis, and urticaria.

In some embodiments, the pruritus is associated with an infectious skin disease selected from fungal disease, bacterial and viral infections, scabies, podiatry, insect bites, and folliculitis.

In some embodiments, the pruritus is associated with an autoimmune skin disease selected from dermatitis herpetiformis (Duhring's disease), bullous pemphigoid, genetic skin disease, Darier's disease, and Hailey-Hailey disease.

In some embodiments, the pruritus is associated with a pregnancy-related skin disease selected from polymorphic eruption of pregnancy (PEP), atopic eruption of pregnancy, pemphigoid gestationis, neoplasia, and cutaneous T-cell lymphoma.

In some embodiments, the pruritus is associated with prurigo nodularis.

In some embodiments, the pruritus is associated with renal disease or a process for the treatment of renal disease.

In some embodiments, the pruritus is associated with chronic renal disease.

In some embodiments, the pruritus is associated with a process for the treatment of renal disease, wherein the process for the treatment of renal disease is selected from hemodialysis and peritoneal dialysis.

In some embodiments, the pruritus is associated with a medical procedure or treatment.

In some embodiments, the pruritus is associated with medical treatment with a drug selected from opioids, antimalarial drugs, anticancer therapies, and epidermal growth factor receptor inhibitors.

In some embodiments, the disease mediated by P2X3 or related to P2X3 activity is endometriosis. Symptoms related to the endometriosis are selected from dysmenorrhea, dyspareunia, dysuria, and schizophrenia.

The present disclosure also provides a use of the pharmaceutical composition or preparation in the treatment and/or prevention of a disease.

The present disclosure also provides a use of the pharmaceutical composition or preparation for preventing, curing, treating, or alleviating a disease in animals (e.g., humans) that is at least partially mediated by P2X3 or related to P2X3 activity. The disease includes, but is not limited to, respiratory disease, cough, chronic cough, idiopathic pulmonary fibrosis, chronic pulmonary obstruction, asthma, pain, urinary incontinence, autoimmune disease, overactive bladder, dysuria, inflammation, Alzheimer's disease, Parkinson's disease, sleep disorder, epilepsy, mental illness, arthritis, neurodegeneration, traumatic brain injury, myocardial infarction, rheumatoid arthritis, stroke, thrombosis, atherosclerosis, colon syndrome, inflammatory bowel disease, digestive tract disease; gastrointestinal dysfunction, respiratory failure, sexual dysfunction, cardiovascular system disease, heart failure, hypertension, urinary incontinence, cystitis, arthritis, endometriosis, blood disease, musculoskeletal and connective tissue developmental disorder, and systemic disorder.

In some embodiments, the disease comprises pain; and the pain includes, but is not limited to: inflammatory pain, surgical pain, visceral pain, dental pain, premenstrual pain, central pain, pain due to burns, migraine, or cluster headache.

In some embodiments, the disease comprises a urinary tract disease.

In some embodiments, the disease comprises a respiratory disease including, but not limited to, a respiratory disorder including idiopathic pulmonary fibrosis, chronic obstructive pulmonary disease, asthma, bronchospasm, or chronic cough.

The chronic cough is a cough that lasts for more than eight weeks and causes serious adverse social, psychological, and physical effects.

In some embodiments, the disease comprises pruritus associated with an inflammatory skin disease, an infectious skin disease, an autoimmune skin disease, or a pregnancy-related skin disease.

In some embodiments, the pruritus is associated with an inflammatory skin disease selected from atopic dermatitis, allergic contact dermatitis, irritant contact dermatitis, xerotic dermatitis, dysplastic dermatitis, lichen planus, lichen sclerosus, polymorphic eruption psoriasis, Graves disease, mucoid degeneration, mastocytosis, and urticaria.

In some embodiments, the pruritus is associated with an infectious skin disease selected from fungal disease, bacterial and viral infections, scabies, podiatry, insect bites, and folliculitis.

In some embodiments, the pruritus is associated with an autoimmune skin disease selected from dermatitis herpetiformis (Duhring's disease), bullous pemphigoid, genetic skin disease, Darier's disease, and Hailey-Hailey disease.

In some embodiments, the pruritus is associated with a pregnancy-related skin disease selected from polymorphic eruption of pregnancy (PEP), atopic eruption of pregnancy, pemphigoid gestationis, neoplasia, and cutaneous T-cell lymphoma.

In some embodiments, the pruritus is associated with prurigo nodularis.

In some embodiments, the pruritus is associated with renal disease or a process for the treatment of renal disease.

In some embodiments, the pruritus is associated with chronic renal disease.

In some embodiments, the pruritus is associated with a process for the treatment of renal disease, wherein the process for the treatment of renal disease is selected from hemodialysis and peritoneal dialysis.

In some embodiments, the pruritus is associated with a medical procedure or treatment.

In some embodiments, the pruritus is associated with medical treatment with a drug selected from opioids, antimalarial drugs, anticancer therapies, and epidermal growth factor receptor inhibitors.

In some embodiments, the disease mediated by P2X3 or related to P2X3 activity is endometriosis. Symptoms related to the endometriosis are selected from dysmenorrhea, dyspareunia, dysuria, and schizophrenia.

The present disclosure also provides a method for treating or preventing a disease by administering a prophylactically and/or therapeutically effective amount of the pharmaceutical composition or preparation to a patient.

By administering the pharmaceutical composition, the side effects of dysgeusia associated with the treatment are reduced.

### Definition and Explanation of Terms

Various terms and phrases used in the present disclosure have general meanings known to those skilled in the art. Even so, these terms and phrases are expected to be described and explained in greater detail herein. If any of the terms and phrases referred to are inconsistent with the known meanings, the meanings expressed in the present disclosure shall prevail.

A polymorph of the compound of formula A in the present disclosure includes crystal forms of non-solvate (anhydrate) and solvate of the compound of formula A.

The polymorph of the compound of formula A in the present disclosure has have characteristic peaks in X-ray powder diffraction expressed at 2θ angles, wherein “±0.20” is the allowable measurement error range.

The polymorph of the compound of formula A in the present disclosure can be used in combination with other active ingredients, provided that it does not produce other adverse effects, such as allergic reactions.

As used herein, the term “composition” is meant to include a product containing a specified amount of each of the specified ingredients, as well as any product derived directly or indirectly from a combination of the specified amount of each of the specified ingredients.

Those skilled in the art can prepare the polymorph of the compound of formula A in the present disclosure into a suitable pharmaceutical composition using a known pharmaceutical carrier. The pharmaceutical composition may be specially formulated for oral administration, for parenteral injection, or for rectal administration in solid or liquid form. The pharmaceutical composition may be formulated into a variety of dosage forms for ease of administration, such as oral preparations (e.g., tablets, capsules, solutions, or suspensions); and injectable preparations (e.g., injectable solutions or suspensions, or injectable dry powders that can be used immediately after adding a drug solvent before injection).

As used herein, the term “therapeutically and/or prophylactically effective amount” refers to the amount of a drug or pharmaceutical preparation that elicits a biological or medical response in a tissue, system, animal, or human sought by a researcher, veterinarian, physician, or other person.

When used for therapeutic and/or prophylactic purposes as described above, the total daily dose of the compound of formula A (including its crystal form) and the pharmaceutical composition in the present disclosure must be determined by the attending physician within the scope of reliable medical judgment. For any specific patient, the specific therapeutically effective dose level depends on a variety of factors, including a disorder being treated and the severity of the disorder; activity of a specific compound used; a specific composition used; age, weight, general health status, sex and diet of the patient; administration time, administration route and excretion rate of the specific compound used; duration of treatment; drugs used in combination or simultaneously with the specific compounds used; and similar factors known in the medical field. For example, it is a common practice in the art to start with a dose of the compound below the level required to obtain the desired therapeutic effect and gradually increase the dose until the desired effect is obtained.

### Beneficial Effects

The pharmaceutical compositions and preparations of the present disclosure have good safety and/or stability, as well as high P2X3 antagonistic activity with less impact on the taste. It has been found that crystal form I, crystal form III, and crystal form IV of the active ingredient in the pharmaceutical composition of the present disclosure have good solid-state properties, wherein crystal form III has good physicochemical properties, and physical and chemical stability; crystal form III has a better PK advantage compared with crystal form I and crystal form IV, and there is no significant difference between the solubility of crystal form III in biological media and that of crystal form I and crystal form V; the crystal form III is less likely to cause risk of crystal transformation during storage and production, and it is easy to prepare, suitable for industrialized scale-up production, especially in the development of solid preparations, which is conducive to maintaining the stability, effectiveness, safety, and quality controllability of API during the preparation process, and has a better prospect of medicinal use.

### BRIEF DESCRIPTION OF THE DRAWINGS

FIG. 1 shows a DSC pattern of amorphous form;

FIG. 2 shows a TGA pattern of amorphous form;

FIG. 3 shows an XRPD pattern of crystal form I;

FIG. 4 shows a TGA/DSC superposition pattern of crystal form I;

FIG. 5 shows an PLM pattern of crystal form I;

FIG. 6 shows an XRPD pattern of crystal form II;

FIG. 7 shows a ¹HNMR spectrum of crystal form II;

FIG. 8 shows a TGA/DSC superposition pattern of crystal form II;

FIG. 9 shows an PLM pattern of crystal form II;

FIG. 10 shows an XRPD pattern of crystal form III;

FIG. 11 shows a TGA/DSC superposition pattern of crystal form III;

FIG. 12 shows an PLM pattern of crystal form III;

FIG. 13 shows an XRPD pattern of crystal form IV;

FIG. 14 shows a TGA/DSC superposition pattern of crystal form IV;

FIG. 15 shows an PLM pattern of crystal form IV;

FIG. 16 shows an XRPD pattern of crystal form V;

FIG. 17 shows a TGA/DSC superposition pattern of crystal form V;

FIG. 18 shows an PLM pattern of crystal form V;

FIG. 19 shows an XRPD pattern of crystal form VI;

FIG. 20 shows an XRPD pattern of crystal form VII;

FIG. 21 shows a ¹HNMR spectrum of residual ethylene glycol of crystal form VII;

FIG. 22 shows a TGA/DSC superposition pattern of crystal form VII;

FIG. 23 shows an XRPD pattern of crystal form VIII;

FIG. 24 shows a TGA/DSC superposition pattern of crystal form VIII;

FIG. 25 shows a ¹HNMR spectrum of crystal form VIII and the raw material;

FIG. 26 shows a TGA/DSC superposition pattern of crystal form IX;

FIG. 27 shows a ¹HNMR spectrum of crystal form IX;

FIG. 28 shows an XRPD pattern of crystal form IX;

FIG. 29 shows an XRPD superposition pattern of crystal form I under ambient humidity, low humidity, and high humidity conditions;

FIG. 30 shows DVS test results of crystal form I;

FIG. 31 shows the results of an XRPD superposition pattern of crystal form I before and after the DVS test;

FIG. 32 shows an XRPD superposition pattern of crystal form III before and after dehydration by heating;

FIG. 33 shows DVS test results of crystal form III;

FIG. 34 shows an XRPD superposition pattern of crystal form III before and after a DVS test;

FIG. 35 shows an XRPD superposition pattern of crystal form IV before and after dehydration;

FIG. 36 shows an XRPD superposition pattern of crystal form I, crystal form VI, and crystal form VI placed under ambient humidity for a few minutes;

FIG. 37 shows an XRPD superposition pattern of crystal form VIII and a dried sample;

FIG. 38 shows an XRPD superposition pattern of the stability test sample of crystal form I;

FIG. 39 shows TGA results of crystal form I after 7 days in a 60° C. (closed) stability test chamber;

FIG. 40 shows an XRPD superposition pattern of the stability test sample of crystal form V;

FIG. 41 shows an XRPD superposition pattern of the stability test (7 days) sample of crystal form III;

FIG. 42 shows an XRPD superposition pattern of the stability test (3 months) sample of crystal form III;

FIG. 43 shows a graph of solubility data of crystal forms I, III, and V;

FIG. 44 shows an XRPD superposition pattern of the remaining solid in the solubility test of crystal form I;

FIG. 45 shows an XRPD superposition pattern of the remaining solid in the solubility test of crystal form V;

FIG. 46 shows an XRPD superposition pattern of the remaining solid in the solubility test of crystal form III;

FIG. 47 shows a schematic diagram of a running procedure for water absorption and dehydration in a dynamic vapor sorption (DVS) test of crystal form III;

FIG. 48 shows a DVS curve of the dynamic vapor sorption (DVS) test of crystal form III;

FIG. 49 shows an XRPD superposition pattern of crystal form III before and after the dynamic vapor sorption (DVS) test;

FIG. 50 shows the in situ test results of variable humidity XRPD of crystal form III;

FIG. 51 shows the in situ test results of variable humidity XRPD of crystal form III (partial enlargement);

FIG. 52 shows an XRPD superposition pattern of the crystal form in the preparation process;

FIG. 53 shows an XRPD superposition pattern of the crystal form of a coated tablet, API, and a blank excipient;

FIG. 54 shows the crystal form stability results of 100 mg tablet cores;

FIG. 55 shows the crystal form stability results of 100 mg coated tablets.

### DETAILED DESCRIPTION OF THE PREFERRED EMBODIMENT

The technical solutions of the present disclosure will be further described in detail below in conjunction with specific examples. It should be understood that the following examples are only used to illustrate and explain the present disclosure, and should not be construed as a limitation on the scope of protection of the present disclosure. All techniques realized based on the above contents of the present disclosure are within the scope of protection intended by the present disclosure.

Unless otherwise stated, the raw materials and reagents used in the following examples are commercially available or can be prepared by known methods.

### Experimental Instruments and Parameter Testing:

### XRPD (X-Ray Powder Diffraction)

The solid obtained in the experiment was tested and analyzed for crystal form using PANalytical Empyrean equipped with a PIXcel^{1D} detector. The instrument parameters are as follows: scanning range: 3 to 400 (2θ); step size: 0.013° (2θ); tube voltage: 45 KV; tube current: 40 mA.

### Variable Humidity X-Ray Powder Diffractometer (VH-XRPD)

The crystal form III (batch number: A10230-047P1) sample was tested and analyzed by VH-XRPD, and this part of the work was completed by WuXi AppTec-STAPharmaceutical. The instrument parameters are as follows:

| Instrument number: | PDS-PF-XRD-01 | |
|---|---|---|
| Instrument model: | Bruker D8 Advance | |
| X-ray source: | Cu: K-Alpha (λ = 1.54060 Å) | |
| Tube voltage: | 40 | KV |
| Tube current: | 40 | mA |
| Sample tray rotation speed: | 0 | rpm |
| Scanning range: | 4 to 40° (2θ) | |
| Fixed incident beam: | Primary soller slit: 2.5° | |
| | Divergence slit: 0.6 mm | |
| | Secondary soller slit: 2.5° | |
| | Anti-scattering slit: 7.100 mm | |
| | Detector slit: 10.50 mm | |
| Detector: | PSD LynxEye | |
| Scanning type: | Locked Coupled | |
| Scanning mode: | Continuous scanning | |
| Scanning step size: | 0.02° | |
| Scanning time per step: | 0.6 | seconds |

The humidity change program settings are as follows:

| XRPD test | Humidity | |
|---|---|---|
| humidity point | equilibration | XRPD |
| (% RH) | time (minute) | test point |
| 40 | 60 | 1 |
| | | (onset) |
| 20 | 120 | 2 |
| 15 | 120 | 3 |
| 10 | 120 | 4 |
| 5 | 120 | 5 |
| 0 | 720 or 900 | 6 |
| 10 | 120 | 7 |
| 20 | 120 | 8 |
| 40 | 240 | 9 |
| | | (end) |

### TGA (Thermogravimetric Analysis)

Samples were subjected to thermogravimetric analysis using Discovery TGA 55 (TA Instruments, US). 2 to 3 mg of the sample was placed in a pre-tared open aluminum tray, automatically weighed in a TGA heating furnace, and then heated to 250° C. at a heating rate of 10° C./min under dry N₂ atmosphere.

### DSC (Differential Scanning Calorimetry)

Solid samples were subjected to DSC analysis using TA Instrument Differential Scanning Calorimeter Q200 and Discovery DSC 250. The sample was weighed to record the value, and then placed in the sample chamber. The sample was heated from 25° C. to different endpoint temperatures at a rate of 10° C./min.

### PLM (Polarized Light Microscopy)

A small amount of powder sample was taken and placed on a slide, added dropwise with a small amount of silicone oil to be better dispersed, covered with a coverslip, and then placed on the stage of Polarizing Microscope ECLIPSE LV100POL (Nikon, JPN). The morphology of the sample was observed at an appropriate magnification and photographed.

### Dynamic Vapor Sorption (DVS)

The water vapor adsorption/desorption data of the samples were collected on an adsorption instrument manufactured by ProUmid GmbH & Co. KG, Germany. Conventionally, about 100 mg of sample was taken and placed in a tared sample tray. The weight change of the sample during the humidity change was recorded by the instrument software. The anhydrous crystal forms I and V were tested according to the following parameters.

| | | |
|---|---|---|
| Sample temperature: | 25° | C. |
| Cycle time: | 10 | minutes |
| Minimum balance time: | 50 | minutes |
| Maximum balance time: | 120 | minutes |

| | |
|---|---|
| Weight balance: | 100% |
| Balance condition: | 0.01%/45 minutes |
| Environmental cycle #1: | 0% to 0%, 1 step, |
| | 40° C., 3 hours |
| Environmental cycle #2: | 0% to 90%, 9 steps, 25° C. |
| Environmental cycle #3: | 80% to 0%, 8 steps, 25° C. |
| Adsorption: | 0, 10, 20, 30, 40, 50, 60, 70, 80, 90 |
| Desorption: | 80, 70, 60, 50, 40, 30, 20, 10, 0 |

The crystal form III was tested with an initial humidity of 50% (ambient humidity), and the specific test parameters are shown in the table below:

| | | |
|---|---|---|
| Sample temperature: | 25° | C. |
| Cycle time: | 10 | minutes |
| Minimum balance time: | 50 | minutes |
| Maximum balance time: | 120 | minutes |

| | |
|---|---|
| Weight balance: | 100% |
| Balance condition: | 0.01%/45 minutes |
| Environmental | 50% to 50%, 1 step, 25° C., 3 hours |
| cycle #1: | |
| Environmental | 50% to 90%, 4 steps, 25° C. |
| cycle #2: | |
| Environmental | 80% to 0%, 8 steps, 25° C. |
| cycle #3: | |
| Environmental | 0% to 90%, 9 steps, 25° C. |
| cycle #4: | |
| Environmental | 80% to 50%, 3 steps, 25° C. |
| cycle #5: | |
| Adsorption: | 50, 60, 70, 80, 90 |
| Desorption: | 80, 70, 60, 50, 40, 30, 20, 10, 0 |
| Adsorption: | 0, 10, 20, 30, 40, 50, 60, 70, 80, 90 |
| Desorption: | 80, 70, 60, 50 |

### Hydrogen Nuclear Magnetic Resonance (1H-NMR)

1H-NMR was performed on AVANCE III HD 300 equipped with a SampleXpress 60 auto sampler.

### High Performance Liquid Chromatography (HPLC)

The instrument used for liquid chromatography analysis was Agilent HPLC 1260 series, and the analytical method is shown in the table below.

### HPLC Test Method Used in Solubility Test

| Chromatographic column model | | XBridge Shield RP18, 4.6 * | | | | | |
|---|---|---|---|---|---|---|---|
| | | 150 mm, 3.5 μm | | | | | |
| Column temperature | | 30° C. | | | | | |
| Detector | | DAD (230 nm) | | | | | |
| Flow rate | | 1.0 mL/min | | | | | |
| Injection volume | | 3 μL | | | | | |
| Run time | | 35 minutes | | | | | |
| Diluent | | 0.1% TFA aqueous solution:ACN = 1:1 | | | | | |
| Mobile | A | 0.1% TFA aqueous solution | | | | | |
| phase | B | ACN | | | | | |
| | Time (min) | 0.0 | 2.0 | 20.0 | 30.0 | 30.5 | 35.0 |
| Elution | Mobile phase | 90 | 90 | 50 | 50 | 90 | 90 |
| gradient | A (%) | | | | | | |
| | Mobile phase | 10 | 10 | 50 | 50 | 10 | 10 |
| | B (%) | | | | | | |

### Example 1: Preparation of Compound of Formula A

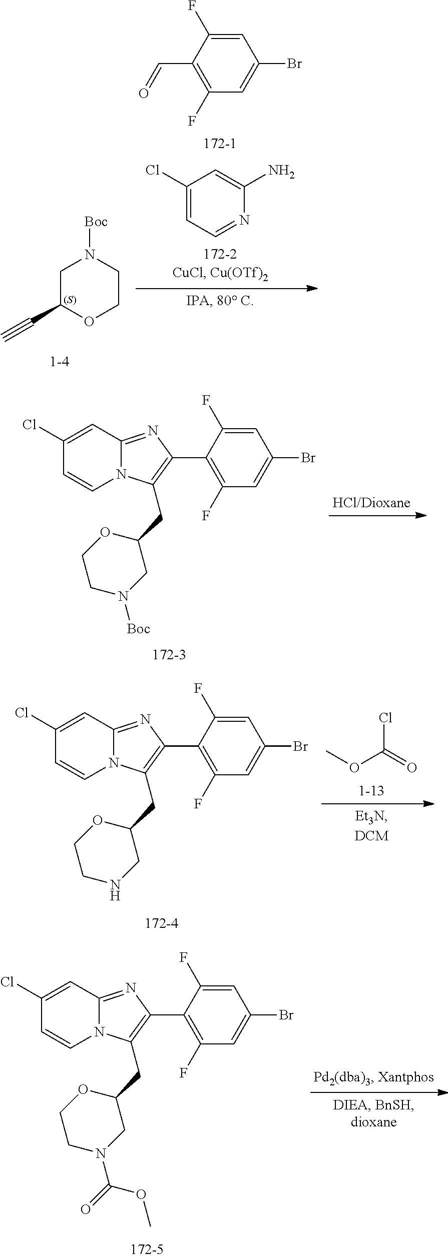

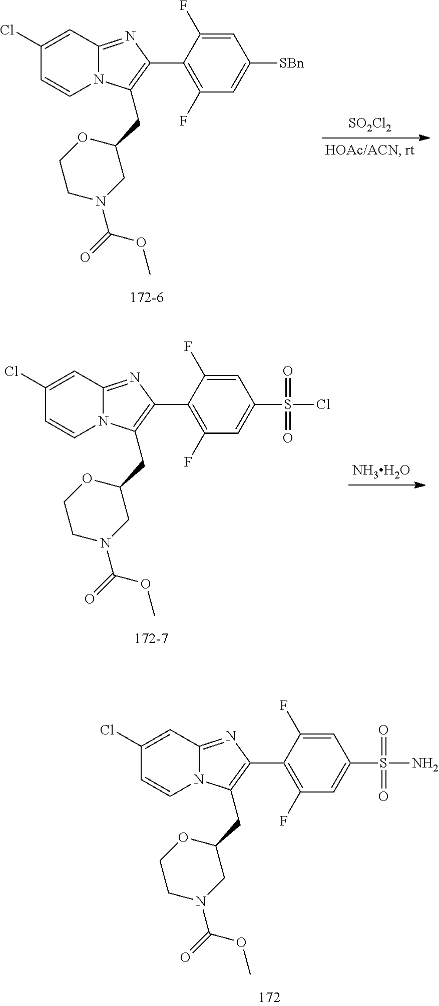

### Step 1: Preparation of tert-butyl (S)-2-((2-(4-bromo-2,6-difluorophenyl)-7-chloroimidazo[1,2-a]pyridin-3-yl)methyl)morpholine-4-carboxylate

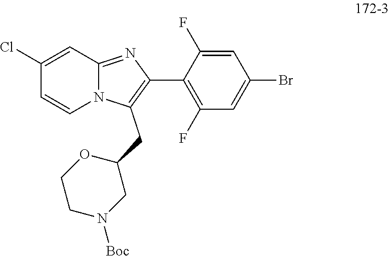 [SMILES: CC(C)(C)OC(=O)N1CCO[C@@H](Cc2c(-c3c(F)cc(Br)cc3F)nc3cc(Cl)ccn23)C1]

To a 100 mL round-bottom flask were sequentially added tert-butyl (S)-2-ethynylmorpholine-4-carboxylate (3.1 g, 1.0 eq, intermediate 1-4), 4-bromo-2,6-difluorobenzaldehyde (2.76 g, 1.0 eq, compound 172-1), 4-chloropyridin-2-amine (1.61 g, 1.0 eq, compound 172-2), CuCl (0.37 g, 0.3 eq), Cu(OTf)₂ (1.36 g, 0.3 eq), and isopropanol (50 mL). The reaction mixture was replaced with nitrogen for three times, heated in an oil bath at 80° C. overnight, and raw material compound 172-2 disappeared, as detected by TLC. The reaction mixture was evaporated to dryness by rotary evaporation to remove isopropanol, and extracted with EA and ammonia water sequentially. The EA phase was washed with saturated brine and citric acid sequentially, dried over anhydrous sodium sulfate, evaporated to dryness by rotary evaporation, and purified by column chromatography to obtain intermediate 172-3 (3.0 g, purity: 78%) as a white solid. LC-MS: [M+H]⁺=542.2.

### Step 2: Preparation of (S)-2-((2-(4-bromo-2,6-difluorophenyl)-7-chloroimidazo[1,2-a]pyridin-3-yl)methyl)morpholine

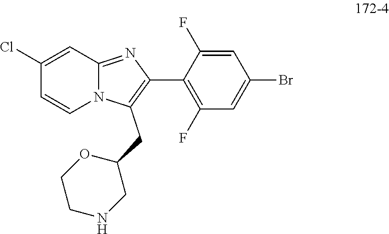 [SMILES: Fc1cc(Br)cc(F)c1-c1nc2cc(Cl)ccn2c1C[C@H]1CNCCO1]

Intermediate 172-3 (2.67 g) was dissolved in dichloromethane (24 mL), and then dioxane hydrochloride (24 mL) was added thereto. The reaction mixture was stirred at room temperature for 1.0 hour, and the reaction was completed, as detected by LC-MS. The reaction mixture was evaporated to dryness by rotary evaporation, added with water (15 mL) and dichloromethane (15 mL), and extracted to obtain an aqueous phase, which was added with sodium bicarbonate aqueous solution to adjust the pH to alkalescence (pH=8 to 9). The phases were separated to obtain a dichloromethane phase, and the aqueous phase was extracted with dichloromethane (10 mL×2). The dichloromethane phases were combined, washed with saturated brine, and evaporated to dryness by rotary evaporation to obtain intermediate 172-4 (1.70 g, purity: 88.6%) as a white solid. LC-MS: [M+H]⁺=442.1.

### Step 3: Preparation of methyl (S)-2-((2-(4-bromo-2,6-difluorophenyl)-7-chloroimidazo[1,2-a]pyridin-3-yl)methyl)morpholine-4-carboxylate

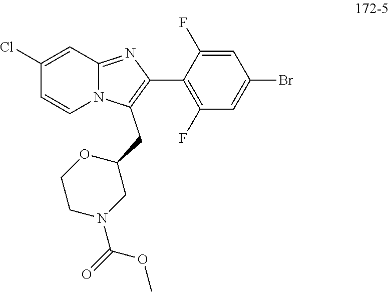 [SMILES: COC(=O)N1CCO[C@@H](Cc2c(-c3c(F)cc(Br)cc3F)nc3cc(Cl)ccn23)C1]

Intermediate 172-4 (1.4 g, 1.0 eq) was dissolved in dichloromethane (10 mL), then triethylamine (480 mg, 1.5 eq) was added thereto, and methyl chloroacetate (388 mg, 1.3 eq) was added dropwise thereto. The reaction mixture was reacted for 1.0 hour, and a product was generated, as shown by LC-MS. After the reaction was completed, the reaction mixture was added with water (10 mL) and stirred for 30 minutes. The phases were separated to obtain a dichloromethane phase, and the aqueous phase was extracted with dichloromethane (10 mL×2). The dichloromethane phases were combined, washed with saturated sodium chloride, dried over anhydrous sodium sulfate, evaporated to dryness by rotary evaporation, and purified by column chromatography to obtain intermediate 172-5 (1.01 g, purity: 93.02%) as a white solid. LC-MS: [M+H]⁺=499.8.

### Step 4: Preparation of methyl (S)-2-((2-(4-(benzylthio)-2,6-difluorophenyl)-7-chloroimidazo[1,2-a]pyridin-3-yl)methyl)morpholine-4-carboxylate

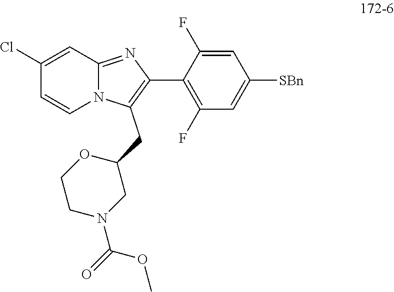

Intermediate 172-5 (0.73 g, 1.0 eq) was dissolved in dioxane (4 mL), and BnSH (0.24 g, 1.3 eq), Pd₂(dba)₃ (0.04 g, 0.03 eq), Xantphos (0.04 g, 0.05 eq), and DIEA (0.60 g, 3.0 eq) were added thereto. The reaction mixture was replaced with nitrogen for three times, and reacted overnight at 80° C. The raw material completely disappeared, as monitored by LC-MS. To the reaction mixture were added dichloromethane (10 mL) and water (10 mL). The phases were separated to obtain a dichloromethane phase, and the aqueous phase was extracted with dichloromethane (10 mL×2). The dichloromethane phases were combined, washed with saturated sodium chloride, dried over anhydrous sodium sulfate, evaporated to dryness by rotary evaporation, and purified by column chromatography to obtain intermediate 172-6 (0.82 g, purity: 91.53%) as a white solid. LC-MS: [M+H]=544.2.

### Step 5: Preparation of methyl (S)-2-((7-chloro-2-(4-(chlorosulfonyl)-2,6-difluorophenyl)imidazo[1,2-a]pyridin-3-yl)methyl)morpholine-4-carboxylate

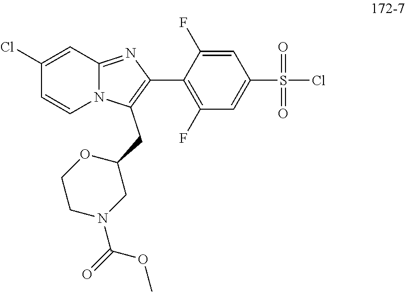 [SMILES: COC(=O)N1CCO[C@@H](Cc2c(-c3c(F)cc(S(=O)(=O)Cl)cc3F)nc3cc(Cl)ccn23)C1]

To a reaction flask was added intermediate 172-6 (510 mg), and acetonitrile (3 mL) was added to dissolve it. The reaction mixture was then added with glacial acetic acid (281 mg, 5.0 eq), and added dropwise with SO₂Cl₂ (506 mg, 4.0 eq) under an ice bath. The reaction mixture was reacted at 0° C. for 1 hour. The raw material disappeared, and intermediate 172-7 was generated, as shown by LC-MS. The reaction mixture was directly used in the next step without treatment.

### Step 6: Preparation of methyl (S)-2-((7-chloro-2-(2,6-difluoro-4-sulfamoylphenyl)imidazo[1,2-a]pyridin-3-yl)methyl)morpholine-4-carboxylate

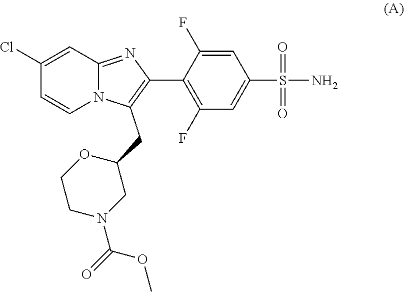 [SMILES: COC(=O)N1CCO[C@@H](Cc2c(-c3c(F)cc(S(N)(=O)=O)cc3F)nc3cc(Cl)ccn23)C1]

To the above reaction mixture was added dropwise ammonia water (2 mL) diluted with acetonitrile (1 mL) at 0° C., and reaction mixture was reacted at room temperature for 0.5 hours. The raw material completely disappeared, and a target product was generated, as shown by LC-MS. The reaction mixture was extracted twice with water and ethyl acetate, washed with brine solution, dried over anhydrous sodium sulfate, concentrated, and purified by C18 chromatography column (water/acetonitrile, RRt=22.5 min). Compound A in an amorphous state (compound A is the compound of formula A) (185 mg, purity: 99.74%) was obtained as a white solid. LC-MS: [M+H]=501.1.

¹H NMR (400 MHz, DMSO-d₆) δ=8.11 (d, J=7.4, 1H), 7.29 (d, J=1.6, 1H), 7.22 (s, 2H), 7.14 (d, J=6.6, 2H), 6.60 (dd, J=7.4, 2.1, 1H), 3.33 (d, J=12.8, 1H), 3.13 (d, J=11.3, 2H), 3.07 (s, 3H), 2.97 (d, J=7.8, 1H), 2.77-2.69 (m, 1H), 2.69-2.61 (m, 1H), 2.53 (dd, J=15.5, 8.3, 1H).

### Post-Processing of Raw Material

A total of 11.1 g of compound A was obtained with reference to the preparation of the compound of formula A, and 20 mL of acetone was added thereto. The reaction mixture was refluxed at 65° C. (under nitrogen atmosphere) for 2.0 hours, directly evaporated to dryness by rotary evaporation to remove acetone, and dried in vacuum at 40° C. for 12 hours. There was about 1% acetone residue, as shown by NMR. The reaction mixture was again dried in vacuum at 80° C. for 12 hours, and there was still acetone residue, as shown by NMR. 5.3 g of the residue was taken and again dried in vacuum at 80° C. for 12 hours, and there was still acetone residue, as shown by NMR, and the resulting product was added with acetonitrile (16 mL). The reaction mixture was refluxed at 85° C. (under nitrogen atmosphere) for 2.0 hours, directly evaporated to dryness by rotary evaporation to remove acetonitrile, and then dried in vacuum at 80° C. for 12 hours. The product was qualified without residual solvent, as shown by NMR, and put in storage for 5.2 g. The product has a purity of 99.29% as a white powder.

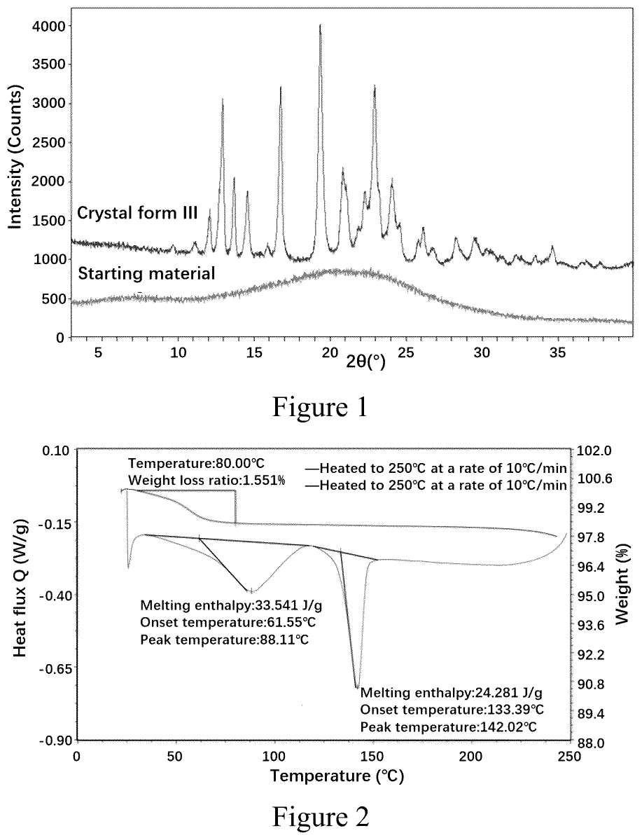

The PLM and XRPD results showed that the raw material is an irregular crystal of 10 to 50 μm with an average crystallinity and an amorphous state. As shown in FIG. 1, the DSC pattern showed that the raw material has two connected endothermic peaks between about 150 to 180° C., with peak temperatures of 164±2° C. and 173±2° C., respectively. As shown in FIG. 2, the TGA pattern showed that the sample substantially has no weight loss before 230° C.

### Example 2: Preparation and Characterization of Amorphous Form

Compound A was dissolved in a certain amount of THF, and concentrated to dryness under reduced pressure to obtain an amorphous sample. The XRPD characterization is as shown in FIG. 1.

### Example 3: Preparation and Characterization of Crystal Form I

3.1 Nine solvents (EtOH, IPA, NBA, MEK, ACN, acetone, EA, IPAc, and Hept) were used respectively, and the mixture was suspended and slurried at room temperature, with a slurrying concentration of 60 mg/mL, to obtain crystal form I;
3.2 six solvents (IPA, NBA, MEK, acetone, Tol, and EA) were used respectively, and the mixture was suspended and slurried at 50° C., with a slurrying concentration of 100 mg/mL, to obtain crystal form I;
3.3 IPAc was selected as the solvent, and the mixture was suspended and slurried at 50° C., with a slurrying concentration of 50 mg/mL, to obtain crystal form I;
3.4 crystal form I can be obtained through slow cooling and crystallization in methanol and ethanol with a cooling temperature from 50° C. to RT;
3.5 THE was used as a good solvent to dissolve the sample to form a sample solution with a certain concentration, and Tol, Hept, and water were slowly added dropwise to the sample solution as an anti-solvent, respectively, at a volume ratio of 1:10 to increase the supersaturation, thereby precipitating the solid to obtain crystal form I;
3.6 EtOH was selected as the solvent for volatile crystallization test to obtain crystal form I.

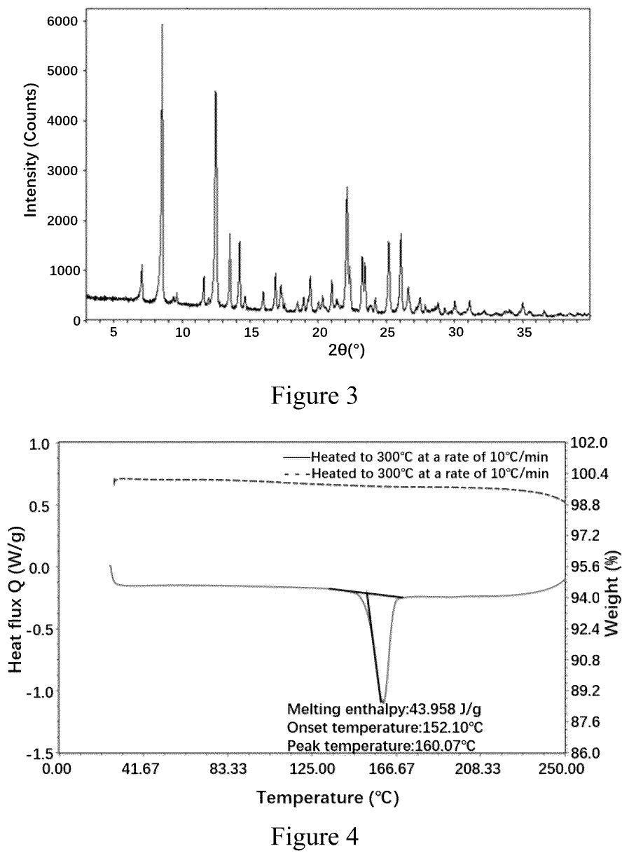

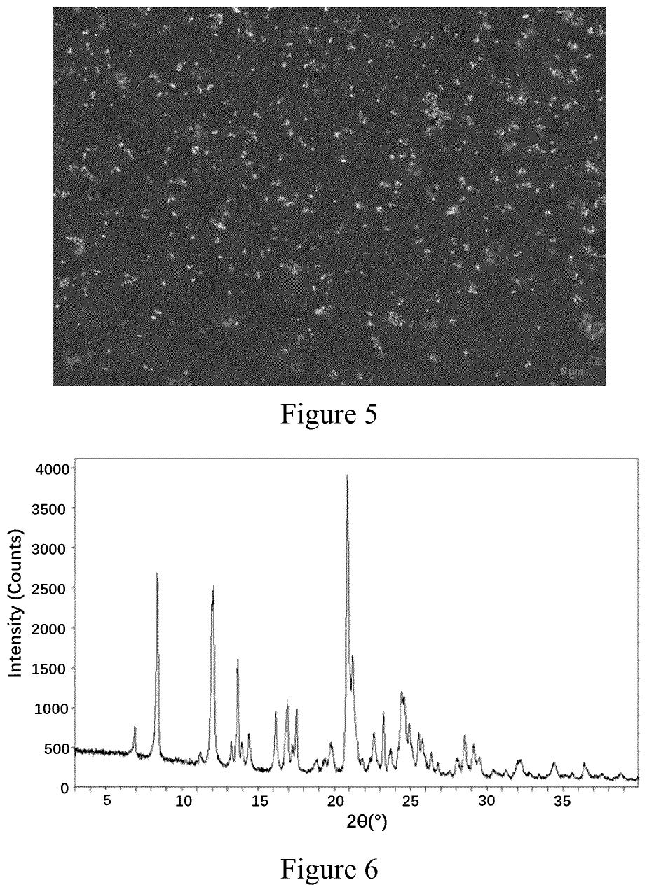

The XRPD pattern of crystal form I expressed at 2θ angles is as shown in FIG. 3. In DSC pattern of the crystal form I, there is an endothermic peak at 152° C. with a melting enthalpy of 44±2 J/g. In TGA pattern of the crystal form I, there is no weight loss in the temperature range of RT to 230° C. The TGA and DSC patterns are as shown in FIG. 4. Combined with the DSC and TGA patterns, it can be seen that the product is an anhydrous crystal form. In PLM pattern of the crystal form I, the crystal form I is an irregular crystal of about 5 μm, and the PLM pattern is as shown in FIG. 5.

### Example 4: Preparation and Characterization of Crystal Form II

4.1 In MTBE, the mixture was suspended and slurried at room temperature to obtain crystal form II;
4.2 MTBE was selected as the solvent, and the mixture was suspended and slurried at 50° C., with a slurrying concentration of 100 mg/mL, to obtain crystal form II;
4.3 THE was used as a good solvent to dissolve the sample to form a sample solution with a certain concentration, and MTBE was slowly added dropwise to the sample solution as an anti-solvent at a volume ratio of 1:10 to increase the supersaturation, thereby precipitating the solid to obtain crystal form II.

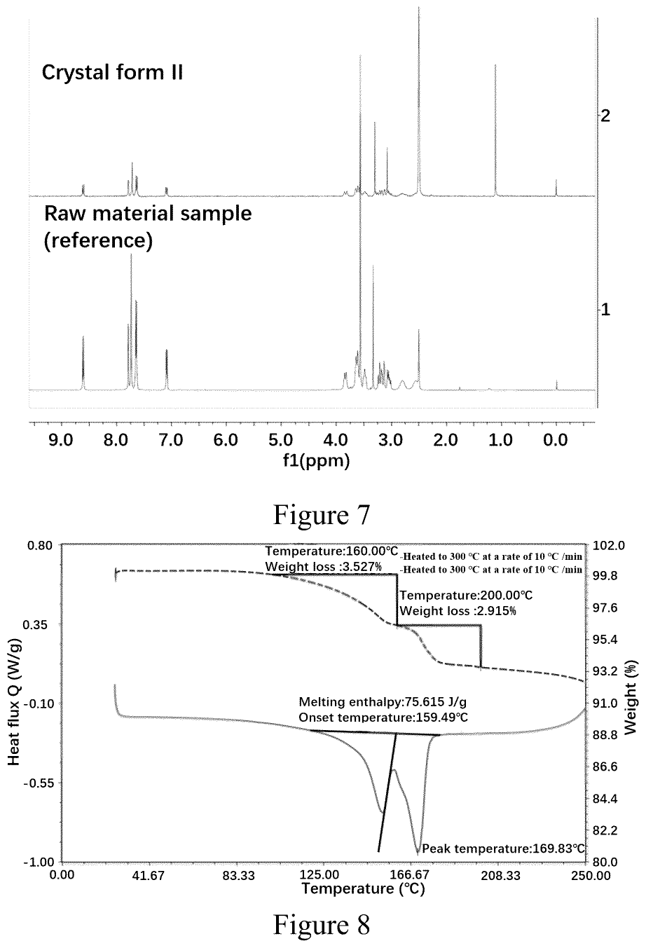

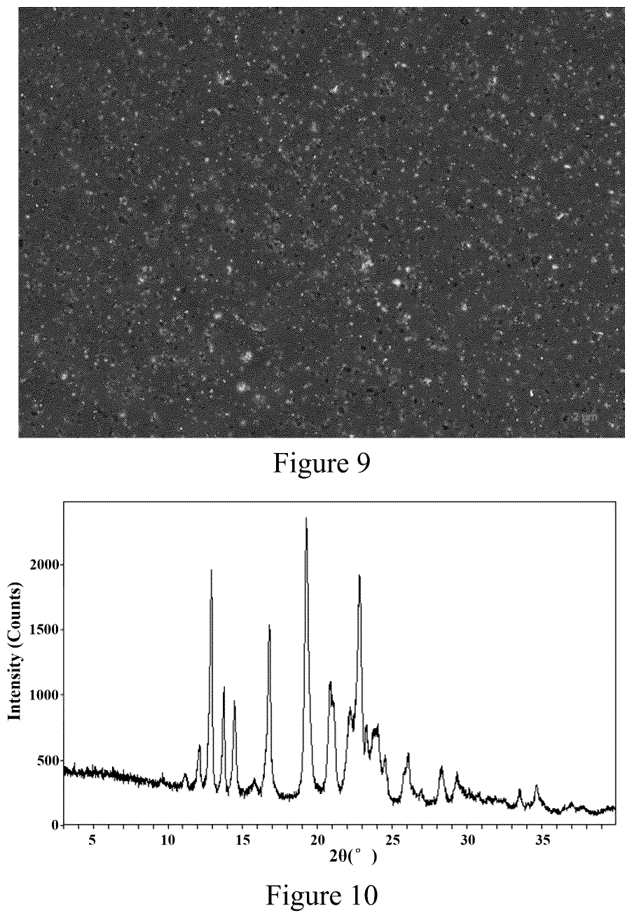

The XRPD pattern of crystal form II expressed at 2θ angles is as shown in FIG. 6. In HNMR spectrum of the crystal form II, there are solvent residual signals of MTBE at the chemical shifts of 1.10 and 3.08 with a molar ratio of 0.39, and the HNMR spectrum of residual MTBE is as shown in FIG. 7. In TGA pattern of the crystal form II, there is a weight loss of 3.5% in the temperature range of 100 to 160° C., and there is a weight loss of 2.9% in the temperature range of 160 to 200° C. In DSC pattern of the crystal form II, there are two adjacent endothermic peaks, and the TGA and DSC patterns are as shown in FIG. 8. Equivalent to about 6.0% of the HNMR spectrum of the residual MTBE as shown in FIG. 7, it can be seen that the product is a MTBE solvate. In PLM pattern of the crystal form II, the crystal form II is an irregular crystal of about 2 μm, and the PLM pattern is as shown in FIG. 9.

### Example 5: Preparation and Characterization of Crystal Form III

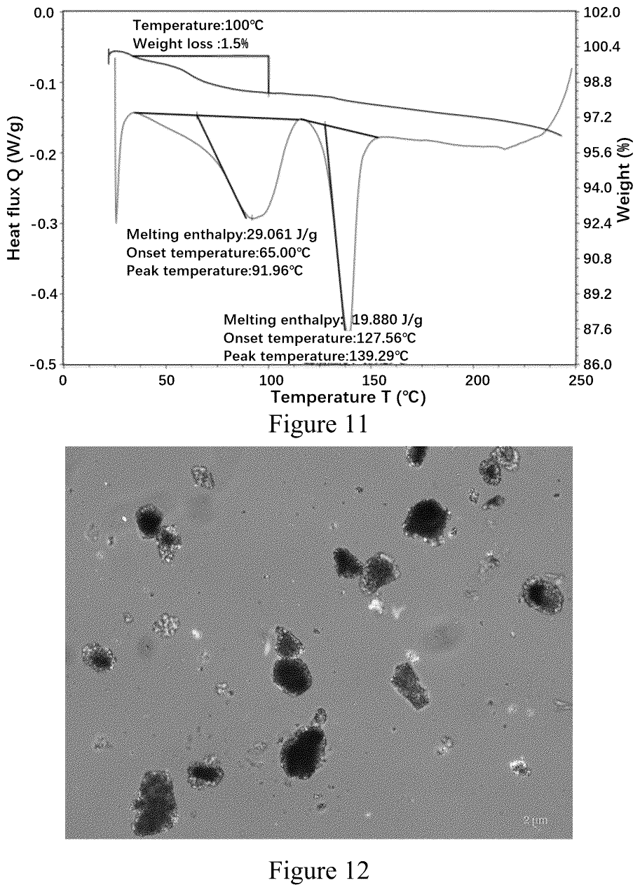

5.1 Water was selected as the solvent, and the mixture was suspended and slurried at 50° C., with a slurrying concentration of 50 mg/mL, to obtain crystal form III.
5.2 The amorphous sample was suspended and slurried in water at 40° C. to obtain crystal form III; specifically, the sample was dissolved in methanol, filtered to obtain a sample solution, and then evaporated by rotary evaporation to obtain 250 mg of an amorphous sample. The amorphous sample was suspended and slurried in water for 20 hours to obtain crystal form III.
5.3 At room temperature, the amorphous sample was dissolved in DMSO and filtered to obtain a DMSO solution, which was slowly added dropwise to water dispersed with crystal form III as a seed crystal, and with the increase of the amount of the DMSO solution added dropwise, the solid precipitation was gradually increased (DMSO/water 1:2, volume ratio). After magnetic stirring at room temperature for 16 hours, the mixture was stopped stirring. The upper liquid was substantially transparent, and the solid quickly settled at the bottom to obtain crystal form III. The XRPD pattern of crystal form III expressed at 2θ angles is as shown in FIG. 10. In TGA pattern of the crystal form III, there is a weight loss gradient of 1.5% in the range of RT to 100° C., and the “%” is the weight percentage. In DSC pattern of the crystal form III, the first endothermic peak is the removal of 0.4 water, and the second endothermic peak is attributed to the melting endothermic peak after sample dehydration, and the TGA and DSC patterns are as shown in FIG. 11. In PLM pattern of the crystal form III, the crystal form III is an irregular crystal of about 2 μm, with an agglomeration of 20 to 50 μm, and the PLM pattern is as shown in FIG. 12.

### Example 6: Preparation and Characterization of Crystal Form IV

The crystal form I product slurry was slurried in 50 to 95% water/acetone (V/V) to obtain crystal form IV

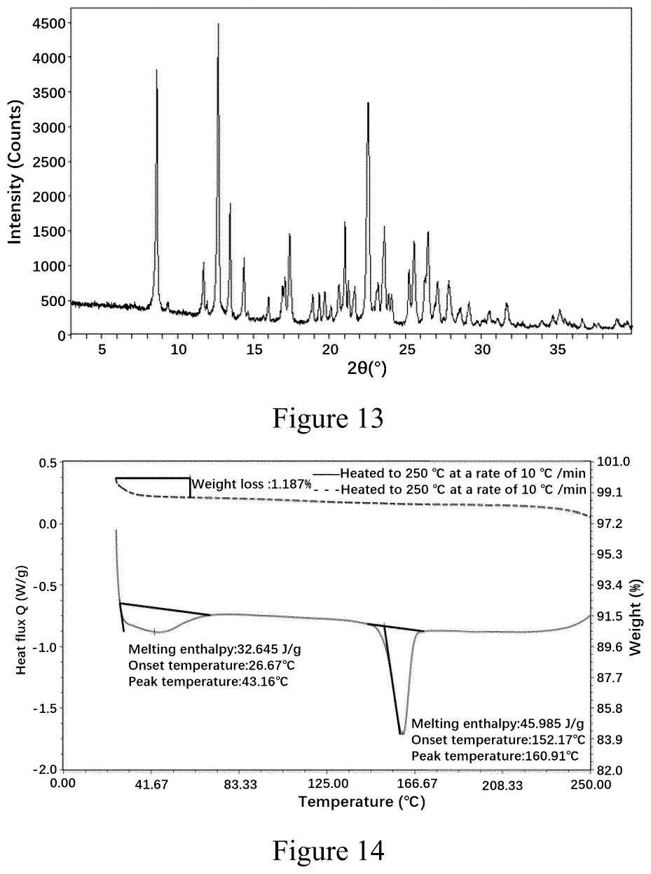

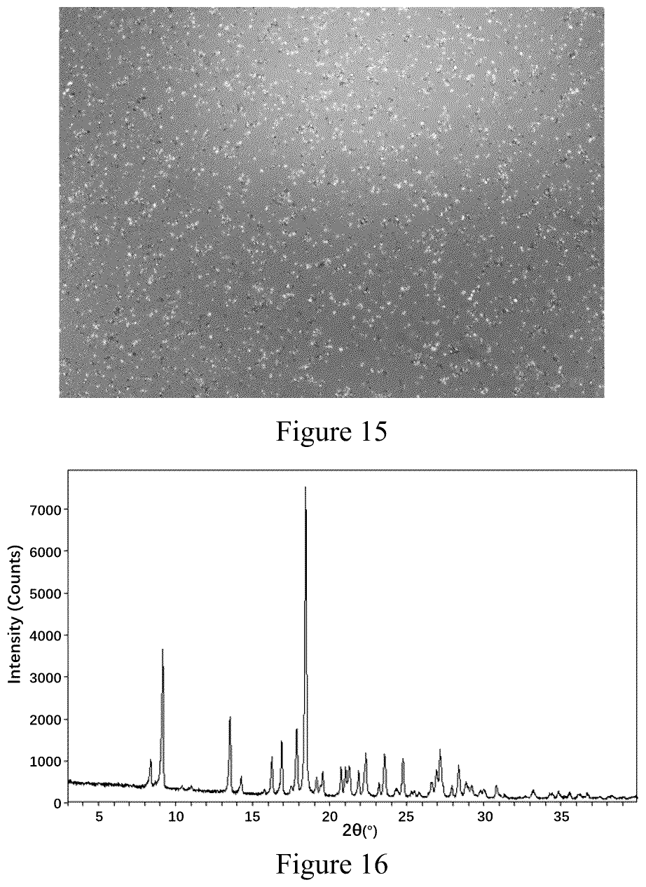

The XRPD pattern of crystal form IV expressed at 2θ angles is substantially as shown in FIG. 13. In TGA pattern of the crystal form IV, there is a weight loss of 1.2% in the temperature range of RT to 60° C. In DSC pattern of the crystal form IV, there are two endothermic peaks, the first broad endothermic peak is presumed to be caused by dehydration, and the subsequent endothermic peak is a melting peak, and the TGA and DSC patterns are as shown in FIG. 14. Combined with the DSC and TGA patterns, it can be seen that the product is a hydrate crystal form with about 0.34 water molecules. In PLM pattern of the crystal form IV, the crystal form IV is an irregular crystal of about 5 μm, and the PLM pattern is as shown in FIG. 15.

### Example 7: Preparation and Characterization of Crystal Form V

7.1 Water was selected as the solvent, and the mixture was suspended and slurried at 50° C., with a slurrying concentration of 50 mg/mL, to obtain crystal form V;
7.2 ACN was selected as the solvent, and the mixture was suspended and slurried at 50° C., with a slurrying concentration of 3.0 mg/mL, to obtain crystal form V;
7.3 MeOH was selected as the solvent for volatile crystallization test to obtain crystal form V.

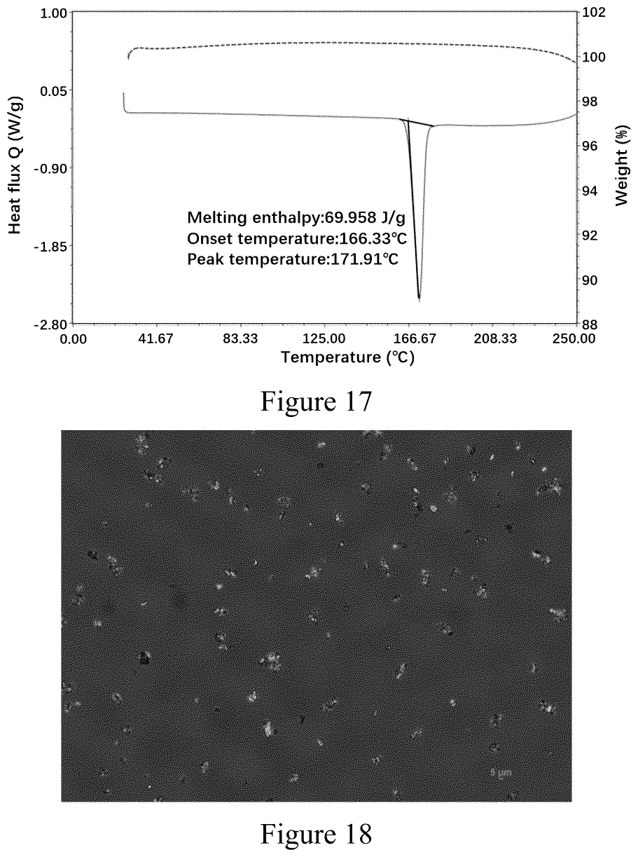

The XRPD pattern of crystal form V expressed at 2θ angles is as shown in FIG. 16. In TGA pattern of the crystal form V, there is no weight loss in the temperature range of RT to 230° C. In DSC pattern of the crystal form II, there is an endothermic peak at 166±2° C. with a melting enthalpy of 70±2 J/g, and the TGA and DSC patterns are as shown in FIG. 17. Combined with the DSC and TGA patterns, it can be seen that the product is an anhydrous crystal form. In PLM pattern of the crystal form V, the crystal form V is an irregular crystal of about 5 μm, and the PLM pattern is as shown in FIG. 18.

### Example 8: Preparation and Characterization of Crystal Form VI

Crystal form VI was obtained by slurrying crystal form I or crystal form V at 60° C. in a mixed solvent of water/acetone with a water content of 10% (volume ratio) at 60° C.

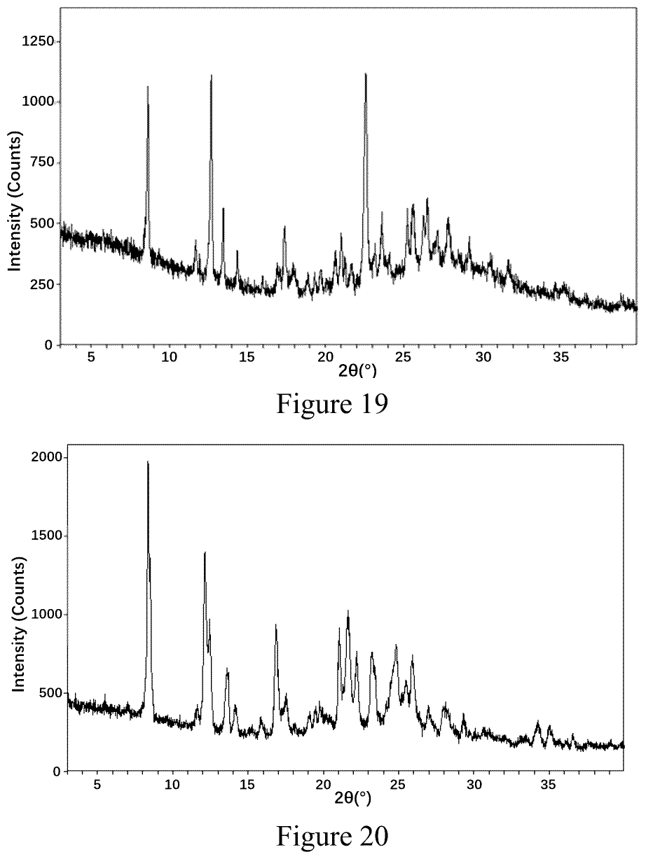

The XRPD pattern of crystal form VI expressed at 2θ angles is as shown in FIG. 19.

### Example 9: Preparation and Characterization of Crystal Form VII

9.1 Ethylene glycol was selected as the solvent, and the mixture was suspended and slurried at 90° C., with a slurrying concentration of 320 mg/mL, to obtain crystal form VII;
9.2 the mixture was slowly cooled and crystallized in ethylene glycol with a cooling temperature from 50° C. to RT to obtain crystal form VII.

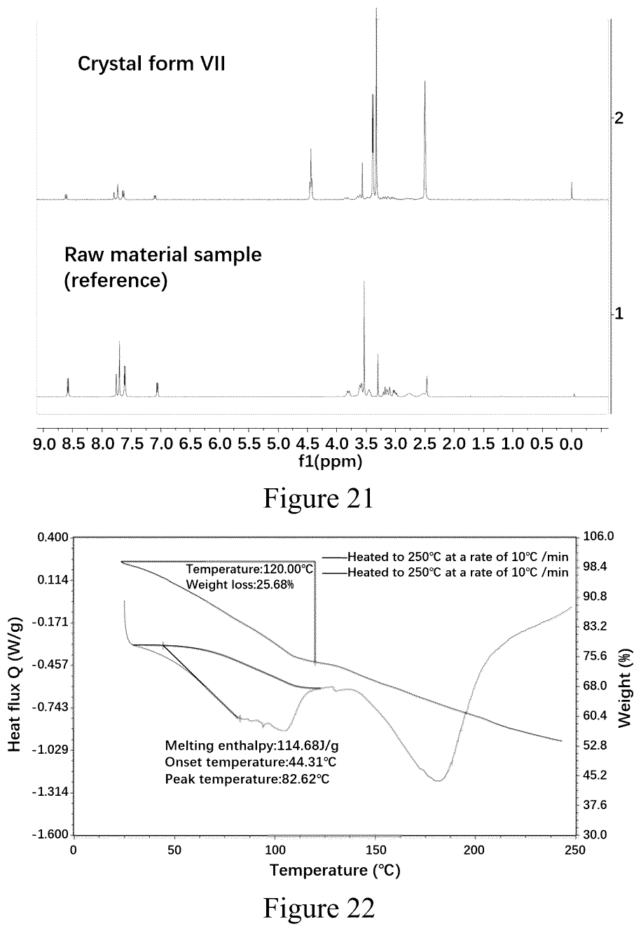

The XRPD of crystal form VII expressed at 2θ angles is substantially as shown in FIG. 20. In ¹HNMR spectrum of the crystal form VII, there is a residual ethylene glycol solvent at the chemical shifts 6 of 3.39 and 4.44, and the ¹HNMR spectrum is as shown in FIG. 21. In TGA pattern of the crystal form VII, there is a weight loss of 25.7% in the range of RT to 120° C. In DSC pattern of the crystal form VII, there are two broad endothermic peaks, the first endothermic peak is presumed to be caused by desolventization, and the TGA and DSC patterns are as shown in FIG. 22. Combined with the DSC and TGA patterns, it can be seen that the product is a solvate containing 2.79 molecules of ethylene glycol.

### Example 10: Preparation and Characterization of Crystal Form VIII

Crystal form III was used as a seed crystal, and water was added dropwise to a saturated solution of 50% THF/water at 40° C. as an anti-solvent to obtain an oil substance. The mixture was cooled to room temperature, and then continued to slurry to obtain crystal form VIII (wet filter cake).

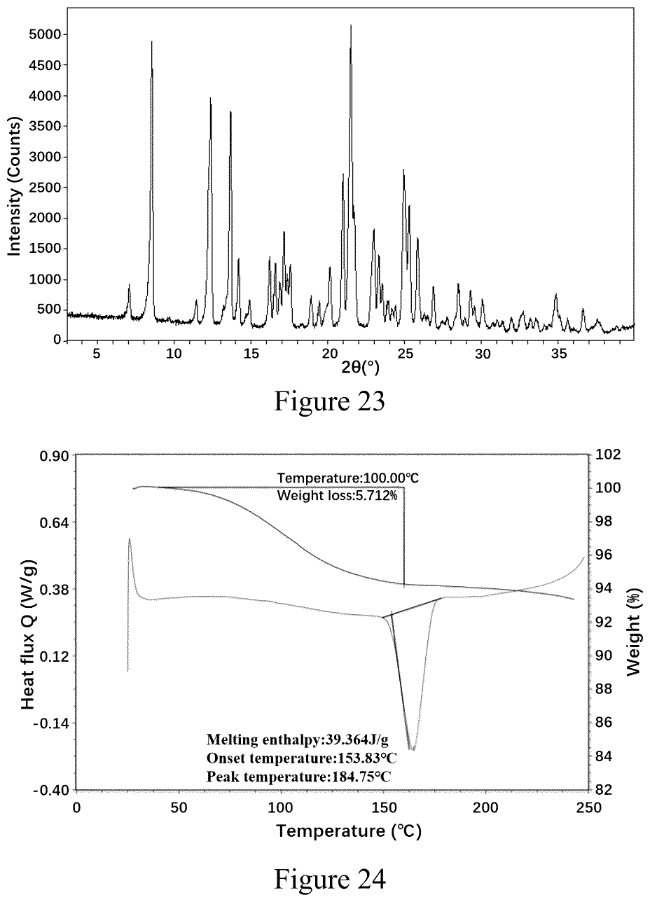

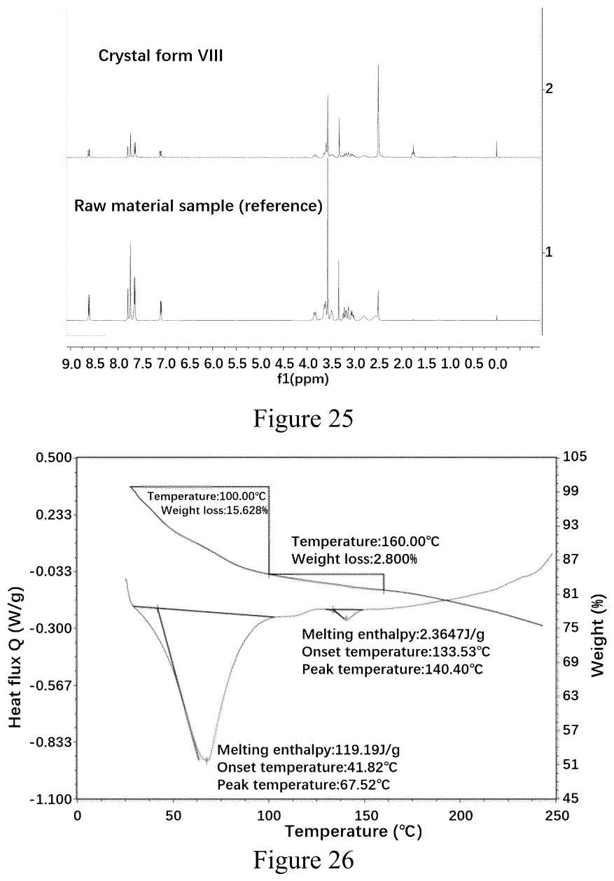

The XRPD pattern of crystal form VIII expressed at 2θ angles is substantially as shown in FIG. 23. In TGA pattern of the crystal form VIII, there is a weight loss of 5.7% in the temperature range of RT to 160° C. In DSC pattern of the crystal form VIII, there is only one endothermic peak, which is the melting peak after desolventization of the sample. Therefore, the crystal form VIII is a solvate, and the TGA and DSC patterns are as shown in FIG. 24. In ¹HNMR spectrum of the crystal form VIII, there is a residual THE solvent at the chemical shifts 6 of 1.76 and 3.60, and the ¹HNMR spectrum is as shown in FIG. 25. The crystal form VIII contains 0.42 molecules of THF.

### Example 11: Preparation and Characterization of Crystal Form IX

Crystal form III was slurried in a saturated solution of 50% DMSO/water at 40° C. to obtain crystal form IX.

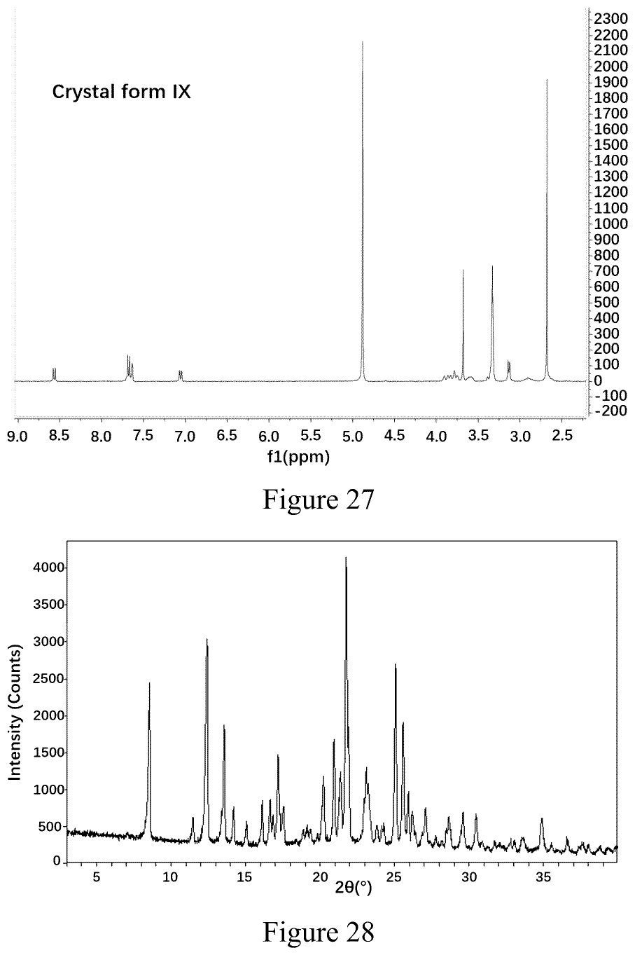

In TGA pattern of the crystal form IX, there is a weight loss of 18.23% in the temperature range of RT to 160° C. In DSC pattern of the crystal form IX, there is a corresponding endothermic peak corresponding to the TGA weight loss on the DSC pattern, and the TGA and DSC patterns are as shown in FIG. 26. In ¹HNMR spectrum of the crystal form IX, there is a residual DMSO solvent at the chemical shift 6 of 2.68, and the ¹HNMR spectrum of residual DMSO is as shown in FIG. 27. Combined with the DSC and TGA patterns, it can be seen that the product is a DMSO solvate. Combined with the DSC and TGA patterns, it can be seen that the product is a DMSO solvate. The XRPD pattern of crystal form IX expressed at 2θ angles is as shown in FIG. 28.

### Example 12: List of XRPD Characteristic Peaks of Crystal Forms of the Present Disclosure

The following table shows data about the positions of the diffraction peaks in the XRPD patterns of nine different crystal forms of the present disclosure whose relative intensity is greater than 10% and whose relative peak intensity ranks among the top ten.

TABLE A
Summary of top ten XRPD peaks of nine different crystal forms

| Form | XRPD Characteristic Peaks/°2θ | | | | | | | | | |
|---|---|---|---|---|---|---|---|---|---|---|
| I | 8.56 | 12.48 | 13.53 | 14.25 | 22.13 | 22.32 | 23.23 | 23.42 | 25.18 | 26.07 |
| II | 8.42 | 12.09 | 13.68 | 16.93 | 17.55 | 20.87 | 21.20 | 22.60 | 23.23 | 24.40 |
| III | 19.27 | 12.91 | 22.80 | 16.77 | 20.86 | 13.75 | 14.46 | 21.08 | 24.05 | 23.75 |
| IV | 8.65 | 12.69 | 13.48 | 14.39 | 17.39 | 21.04 | 22.56 | 23.63 | 25.60 | 26.52 |
| V | 8.38 | 9.15 | 13.52 | 16.24 | 16.89 | 17.84 | 18.43 | 22.33 | 23.55 | 24.74 |
| VI | 23.20 | 20.62 | 20.82 | 13.79 | 24.81 | 11.61 | 22.9 | 11.38 | 21.59 | 23.62 |
| VII | 2.13 | 8.36 | 21.66 | 12.45 | 24.82 | 16.84 | 23.22 | 21.07 | 13.61 | 24.57 |
| VIII | 21.49 | 21.72 | 12.38 | 8.53 | 13.66 | 24.94 | 20.99 | 25.31 | 23.00 | 17.14 |
| IX | 21.75 | 12.43 | 25.07 | 8.55 | 23.12 | 25.57 | 13.57 | 17.18 | 21.37 | 20.94 |

### Example 13: Screening and Performance Studies of Crystal Forms of the Present Disclosure

### 13.1 Summary of Characterization and Performance Studies of Crystal Forms of the Present Disclosure

In order to further develop a form of the compound of formula A of the present disclosure suitable for pharmaceutical preparation, a total of nine different crystal forms were obtained through screening experiments, including two anhydrous crystal forms (crystal form I and crystal form V), three hydrates (crystal form III, crystal form IV, and crystal form VI), and four solvates (crystal form II, crystal form VIII, and crystal form IX). Specific characterization data and performance comparisons are summarized in Table B below. After comparative analysis, it is found that crystal forms I, III, and V have good solid-state properties compared to the other crystal forms and will be subsequently tested for stability and solubility in biological media, respectively.

TABLE B

| | | DSC, endo Onset/Peak | TGA wt loss | DVS (wt %, | |
|---|---|---|---|---|---|
| Crystal form | PLM | (° C.), ΔH (J/g) | %/@T (° C.) | 80%/90% RH) | Remark |
| I | Irregular | 152/159, 44 | about 0/RT to 230 | 6.8, 6.9 | Transformed into crystal |
| Anhydrous | crystal | | | Hygroscopic; | form IV when RH > 40%; risk |
| crystal form | | | | reversible | of crystal transformation |
| | | | | | during storage and production |
| II | Irregular | 152, 170 | 3.5/100 to 160 | N/A | 6.0% MTBE |
| MTBE solvate | crystal | peak temperature | 2.9/160 to 200 | | |
| III | Irregular | 61/88, 34 | 1.6/30 to 80 | 2.7, 2.8 | Low risk of crystal |
| Hemihydrate | crystal | 133/142, 24 | | Hygroscopic; | transformation; careful |
| | | | | reversible | monitoring of water content |
| IV | Irregular | 27/43, 33 | 1.2/RT to 60 | N/A | Transformed into crystal |
| Hydrate | crystal | 152/161, 46) | | | form I after dehydration |
| | | | | | when RH < 60%; risk of |
| | | | | | crystal transformation |
| | | | | | during storage and production |
| V | Irregular | 166/172, 70 | about 0/RT to 230 | 0.7, 0.9 | Can only be obtained by |
| Anhydrous | crystal | | | Slightly | slurrying in high-temperature |
| crystal form | | | | hygroscopic | water, and cannot be obtained |
| | | | | | repeatedly by using other |
| | | | | | batches of raw materials |
| VI | N/A | N/A | N/A | N/A | Extremely unstable; quickly |
| Hydrate | | | | | transformed into crystal |
| | | | | | form I under ambient humidity |
| VII | N/A | 83, 187 (peak | 25.7/RT to 120 | N/A | N/A |
| Ethylene glycol | | temperatures of | | | |
| solvate | | two broad peaks) | | | |
| VIII | N/A | 154/165, 39 | 5.7/40 to 160 | N/A | Unstable; transformed into |
| Hydrate | | | | | crystal form I after dehydration |
| IX | N/A | 42/68, 120 | 15.4/RT to 100 | N/A | N/A |
| DMSO solvate | | 134/140, 2 | 2.8/100 to 160 | | |

### 13.2 Crystal Form I

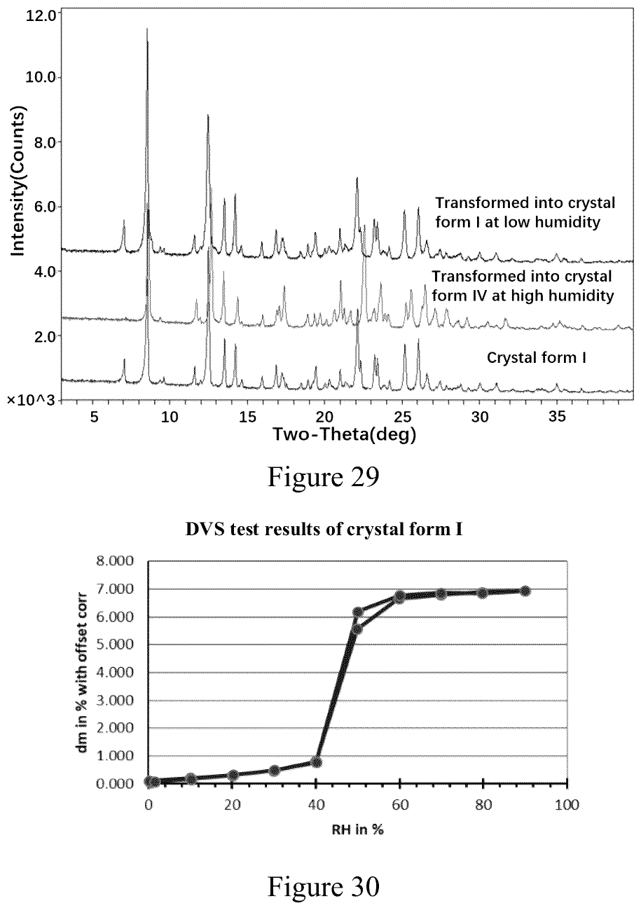

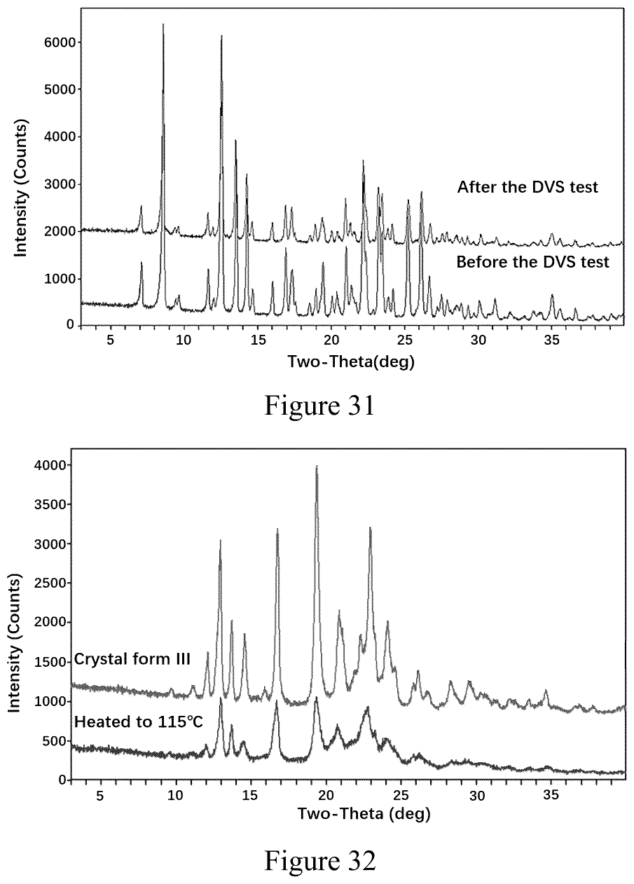

Crystal form I can be obtained by the preparation method in Example 3. It was found that crystal form I absorbed moisture under high humidity environment and was transformed into crystal form IV as a hydrate, but transformed into the initial crystal form I after being dried in a vacuum oven at 30° C. (FIG. 29). Combined with the DVS test results (FIG. 30), it can be seen that the mutual transformation between crystal forms I and IV is reversible. Crystal form I showed hygroscopicity (6.8%, 80% RH), but the crystal form of crystal form I remained unchanged after the DVS test (FIG. 31).

### 13.3 Crystal Form III

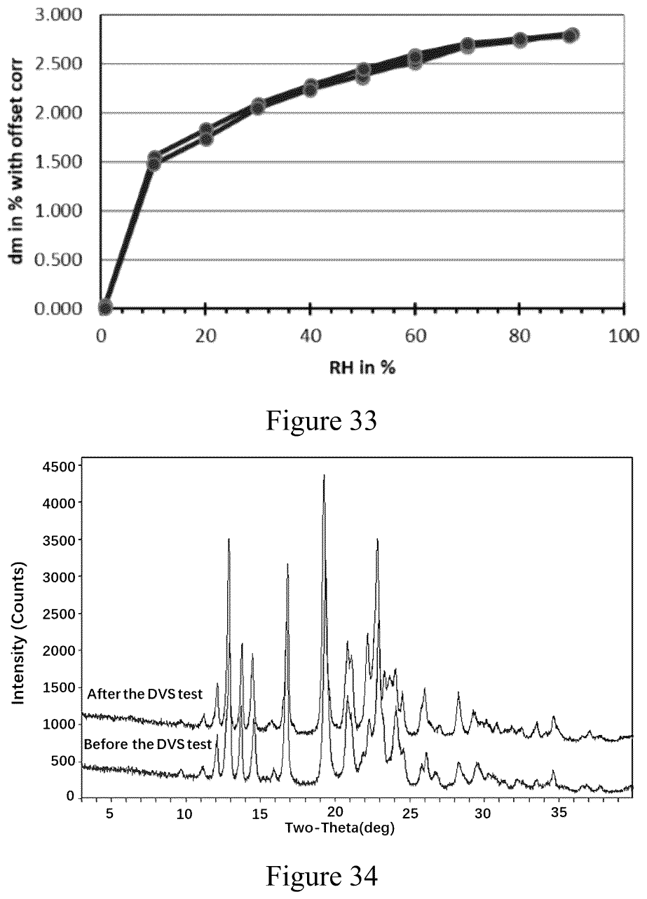

Crystal form III was obtained by the preparation method in Example 5. The experimental results (FIG. 32) showed that after crystal form III was dehydrated, it quickly absorbed moisture under ambient humidity and became crystal form III again. The DVS results (FIG. 33) showed that the crystal form III sample had a sudden change in weight at 10% RH, presumably corresponding to the removal and acquisition of crystal water, respectively; and the water content of the sample changed little in a wide humidity range. As shown in FIG. 34, there was no significant change in XRPD before and after the DVS test.

### 13.4 Crystal Form IV

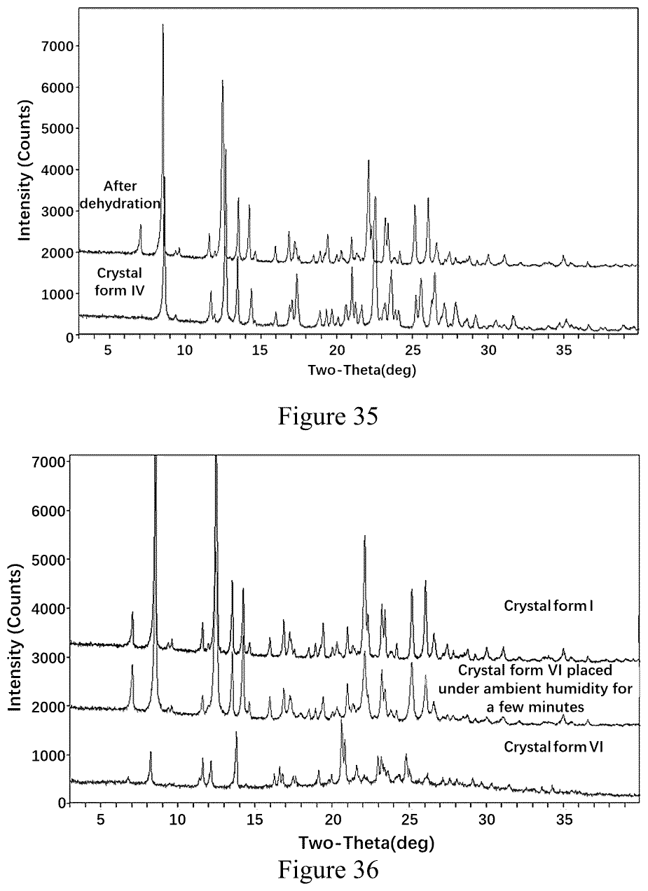

Crystal form IV is a hygroscopic product of crystal form I under high humidity environment, and can be obtained by slurrying crystal form I or crystal form V in a mixed solvent of water/acetone with a water content of 50% to 95%. Crystal form IV was only stable under high humidity environment, and was transformed into crystal form I as an anhydrous crystal form after vacuum drying and dehydration (FIG. 35).

### 13.5 Crystal Form V

Crystal form V is an anhydrous crystal form, which was obtained only by slurrying the first batch of raw materials in water at 60° C. or by suspending and slurrying crystal forms I and V in equal proportions at 60° C., but neither of the preparations could be subsequently repeated.

### 13.6 Crystal Form VI

Crystal form VI was obtained by slurrying crystal form I or crystal form V at 60° C. in a mixed solvent of water/acetone with a water content of 10% (volume ratio) at 60° C. As shown in FIG. 36, the crystal form sample was transformed into crystal form I after being placed under ambient humidity (35% RH) for a few minutes. This indicates that crystal form VI may be an extremely unstable hydrate.

### 13.7 Crystal Form VIII

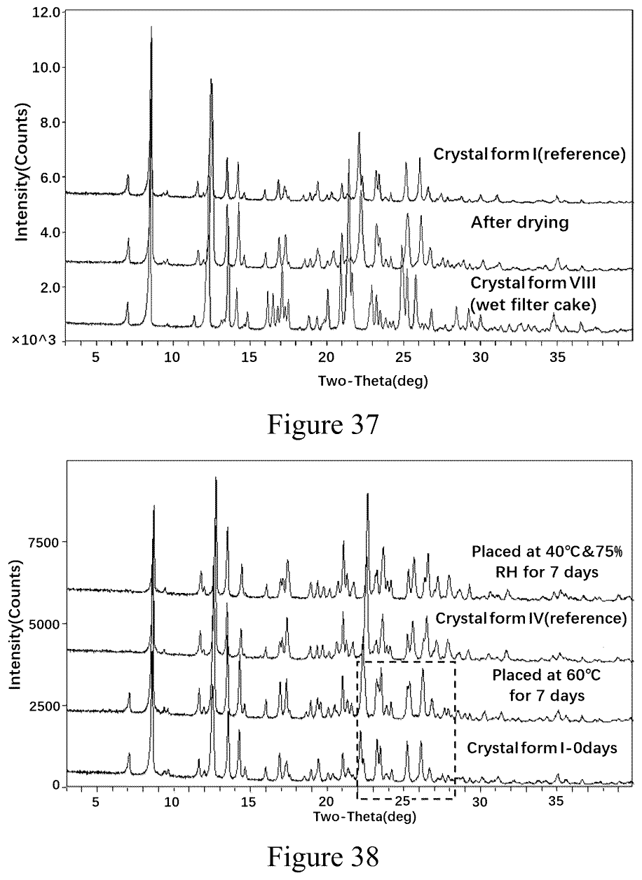

Crystal form VIII is a hydrate, but the hydrate is unstable. After dehydration (vacuum drying at 40° C. for 3 hours), crystal form VIII was transformed into crystal form I (FIG. 37).

### 13.8 Study on Stability of Crystal Forms I, III and V

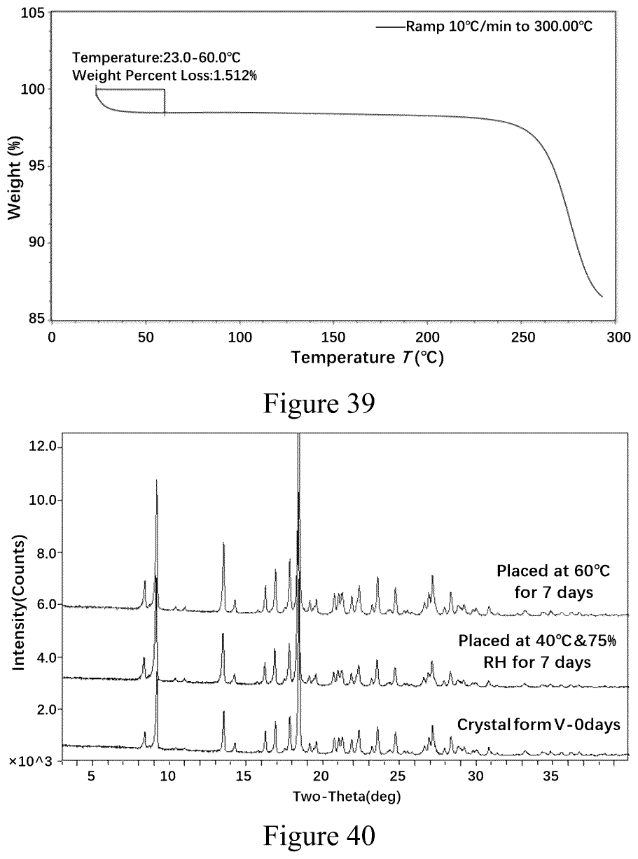

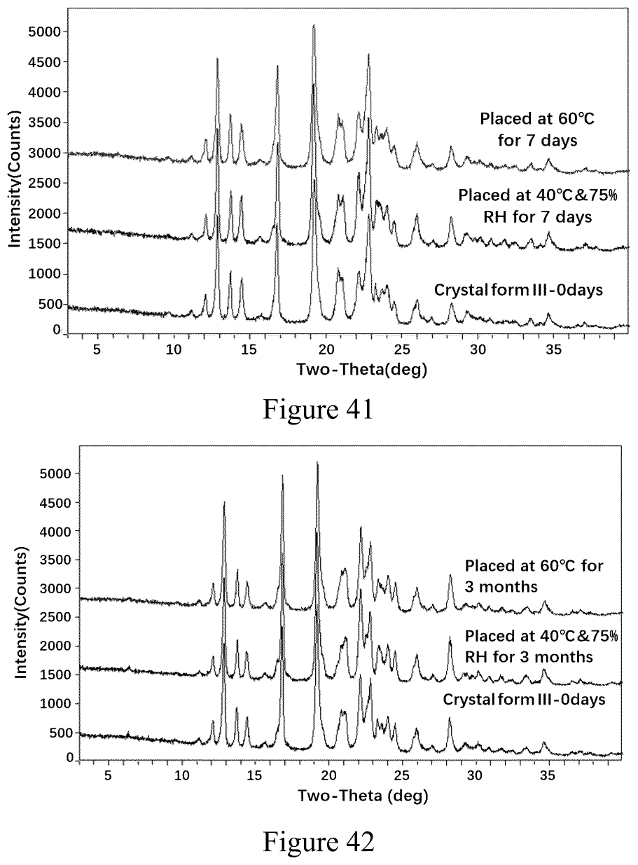

The physical and chemical stability of crystal forms I, III, and V at 60° C. (closed) and 40° C./75% RH (open) for 7 days were investigated respectively. As shown in Table C and FIGS. 38 to 41, crystal form III and crystal form V showed no change in their crystal forms after 7 days under the above test conditions, and there was no significant decrease in chemical purity. This indicates that crystal form III and crystal form V have good physical and chemical stability. As for crystal form I, it absorbed moisture at 40° C./75% RH (open) and was transformed into crystal form IV as a hydrate, which was consistent with the DVS results.

Crystal form III was used as a target crystal form for the next step of development, and the stability of crystal form III was further confirmed. The physical and chemical stability of crystal form III at 60° C. (closed) and 40° C./75% RH (open) for 3 months were investigated. As shown in Table D and FIG. 42, crystal form III showed no change in its crystal form after 3 months under the above test conditions, indicating good physical stability.

TABLE C
Results of stability evaluation of crystal forms I, III, and V

| | | Purity - 7 days | | | |
|---|---|---|---|---|---|
| Sample | Initial | (peak area %) | | XRPD | |
| (batch | purity | 40° C./ | | 40° C./ | |
| number) | (peak area %) | 75% RH | 60° C. | 75% RH | 60° C. |
| Crystal | 99.38 | 99.38 | 99.37 | Crystal | Crystal |
| form I | | | | form IV | form I |
| Crystal | 99.29 | 99.28 | 99.30 | Crystal | Crystal |
| form V | | | | form V | form V |
| Crystal | 99.34 | 99.35 | 99.35 | Crystal | Crystal |
| form III | | | | form III | form III |

TABLE D
Results of 3-month stability evaluation of crystal form III

| | Purity - 1 month | | Purity - 3 months | | | |
|---|---|---|---|---|---|---|
| Initial purity | (peak area %) | | (peak area %) | | XRPD - 3 months | |
| (peak area %) | 40° C./75% RH | 60° C. | 40° C./75% RH | 60° C. | 40° C./75% RH | 60° C. |
| 99.33 | 99.29 | 99.28 | 99.23 | 99.25 | Crystal form III | Crystal form III |

### 13.9 Solubility Test of Crystal Forms I, III, and V in Biological Media

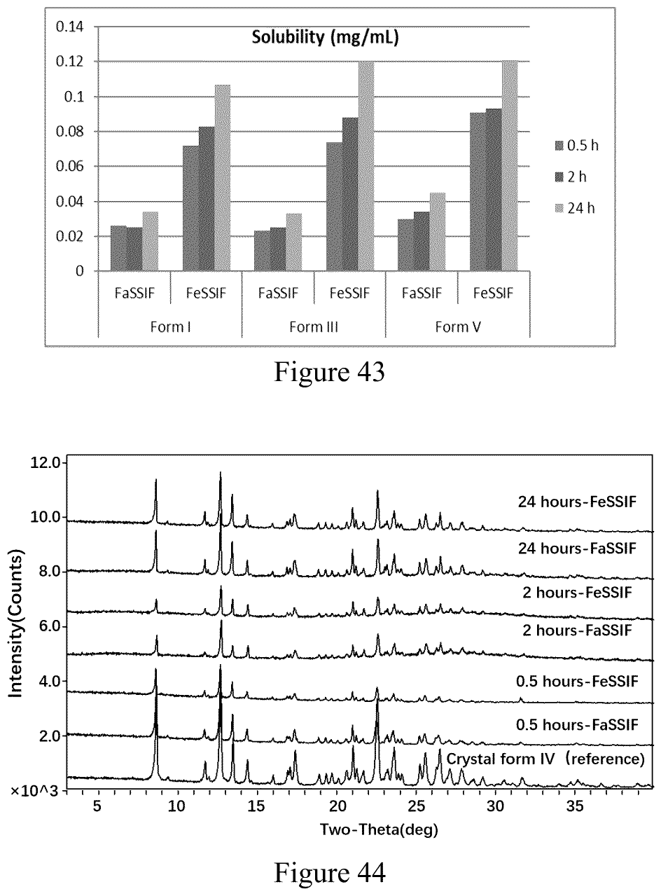

The results of solubility tests of crystal forms I, III, and V in biological media of SGF, FaSSIF, and FeSSF are as shown in Table E and FIG. 43. There was little difference in the solubility of the above three crystal forms in different biological media, and the solubility in SGF was higher than 5 mg/mL. The solubility in FeSSIF was about 3 times that in FaSSIF, indicating that food may contribute to drug absorption. There was no significant change in the pH of biological medium buffers during the test.

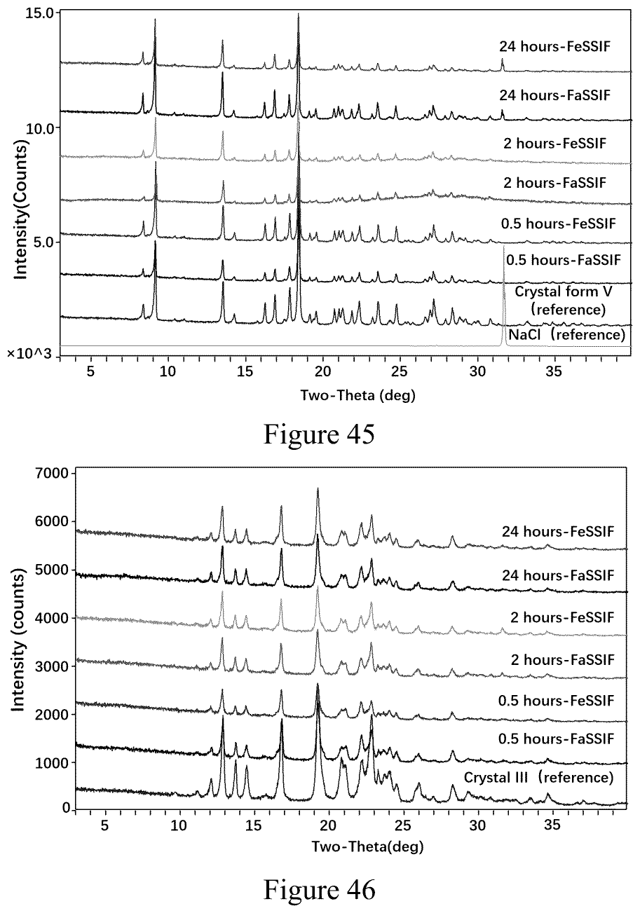

As shown in FIG. 44, crystal form I was transformed into crystal form IV as a hydrate after being placed in FaSSIF and FeSSIF buffers for 0.5 hours. However, crystal forms III and V showed no change in their crystal forms during the test, and the results are as shown in FIG. 45 and FIG. 46.

TABLE E
Results of solubility tests of crystal forms I, III, and V in biological media

| | | Solubility (mg/mL) | | | pH | | | XRPD | | |
|---|---|---|---|---|---|---|---|---|---|---|
| Sample | Medium | 0.5 h | 2 h | 24 h | 0.5 h | 2 h | 24 h | 0.5 h | 2 h | 24 h |
| Crystal | SGF | Dissolved | / | / | 1.2 | 1.3 | 1.2 | N/A | N/A | N/A |
| form I | | clarification | | | | | | | | |
| | FaSSIF | 0.026 | 0.025 | 0.034 | 6.4 | 6.4 | 6.4 | Crystal | Crystal | Crystal |
| | | | | | | | | form IV | form IV | form IV |
| | FeSSIF | 0.072 | 0.083 | 0.107 | 5.0 | 5.1 | 5.1 | Crystal | Crystal | Crystal |
| | | | | | | | | form IV | form IV | form IV |
| Crystal | SGF | 4.331 | Dissolved | / | 1.2 | 1.1 | 1.2 | N/A | N/A | N/A |
| form V | | | clarification | | | | | | | |
| | FaSSIF | 0.030 | 0.034 | 0.045 | 6.4 | 6.4 | 6.4 | Unchanged | Unchanged | Unchanged |
| | FeSSIF | 0.091 | 0.093 | 0.121 | 5.0 | 5.0 | 5.1 | Unchanged | Unchanged | Unchanged |
| Crystal | SGF | Dissolved | / | / | 1.0 | / | / | N/A | N/A | N/A |
| form III | | clarification | | | | | | | | |
| | FaSSIF | 0.023 | 0.025 | 0.033 | 6.4 | 6.4 | 6.4 | Unchanged | Unchanged | Unchanged |
| | FeSSIF | 0.074 | 0.088 | 0.120 | 4.9 | 4.8 | 4.9 | Unchanged | Unchanged | Unchanged |

“Dissolved clarification”: indicates that the solubility of the sample in the medium is greater than 5 mg/mL by visual observation;
“N/A”: indicates no solid sample to test for XRPD.

The results of the above tests show that crystal form III has good physicochemical properties, and physical and chemical stability, and that there is no significant difference between the solubility of crystal form III in biological media and that of crystal form I and crystal form V. Since crystal form I is easy to transform into crystal form IV when RH >40%, there is a risk of crystal transformation during storage and production, and crystal form V is not easy to prepare and is not suitable for scale-up production, crystal form III is more preferred in the present disclosure, which is suitable for preparation development.

### Example 14: Scale-Up Preparation of Crystal Form III

### 1) Solution Preparation:

To reaction kettle A was added DMSO (4 V) under nitrogen atmosphere, and the stirring was started. API was added thereto, and the reaction kettle was rinsed with DMSO (0.8 V). The temperature was adjusted to 25±5° C., and the reaction mixture was stirred for at least 0.5 hours.

### 2) Crystal Transformation:

To reaction kettle B was added purified water (20 V) through a microporous filter, and the temperature was adjusted to 40±5° C. To reaction kettle B was added dropwise DMSO solution in reaction kettle A through a microporous filter for at least 2 hours, and the microporous filter was rinsed with DMSO (0.2 V). The reaction mixture was stirred for at least 4 hours, and a sample was collected for XRPD analysis with a standard of being consistent with the XRPD pattern of the control (crystal form III). If it did not meet the standard, the temperature was controlled at 40±5° C. The reaction mixture was stirred for at least another 4 hours, and a sample was collected for XRPD analysis until it met the standard. The reaction mixture was cooled to 25±5° C. and stirred for at least 2 hours. The reaction mixture was centrifuged with a 300 mesh filter cloth bag, and the filter cake was rinsed with purified water (2 V) and collected. To reaction kettle B was added purified water (10 V) through a microporous filter under nitrogen atmosphere, and the stirring was started. The above wet filter cake was added thereto, and the temperature was controlled at 25±5° C. The reaction mixture was stirred for at least 0.5 hours, centrifuged with a 300 mesh filter cloth bag, and the filter cake was rinsed with purified water (5 V) and collected.

### 3) Drying:

The filter cake was dried in a vacuum oven at 45±5° C. for at least 4 hours, and a sample was collected for GC with a standard of DMSO ≤0.5000%. If it did not meet the standard, a sample was collected for GC analysis at least every 2 hours until it met the standard. A sample was collected for KF with a standard of 2.0% to 3.0%. If KF was higher than 3.0%, the reaction mixture was dried at 45±5° C. for at least 1 hour, and a sample was collected for KF until it met the standard. If KF was lower than 2.0%, wet nitrogen stream was blown into the oven for at least 0.5 hours, and a sample was collected for KF until it met the standard.

The basic information of the prepared multiple batches of products is summarized in Table F:

TABLE F
Summary of moisture and particle size data for each batch

| | Production | | | Moisture | Particle size | | |
|---|---|---|---|---|---|---|---|
| Batch | per batch | | Crystal form | (%) | D₁₀ | D₅₀ | D₉₀ |
| 1 | 1.1844 | kg | Crystal form III | 2.2% | 2.05 | 10.8 | 23.0 |
| 2 | 1.2824 | kg | Crystal form III | 2.4% | 1.33 | 7.99 | 17.0 |
| 3 | 1.612 | kg | Crystal form III | 2.8% | 1.67 | 11.1 | 24.2 |

### Example 15: Study on Stability of Crystal Form III Preparation

In specific preparation formulation and preparation process, the crystal form is often prone to crystal transformation, which leads to changes in drug properties, compatibility, etc., which in turn has an impact on the stability, effectiveness, safety, quality controllability, etc. of the preparation. Therefore, it is necessary to further study the stability performance of crystal form III in the preparation of the present disclosure.

### 15.1 Dynamic Vapor Sorption (DVS) Test of Crystal Form III

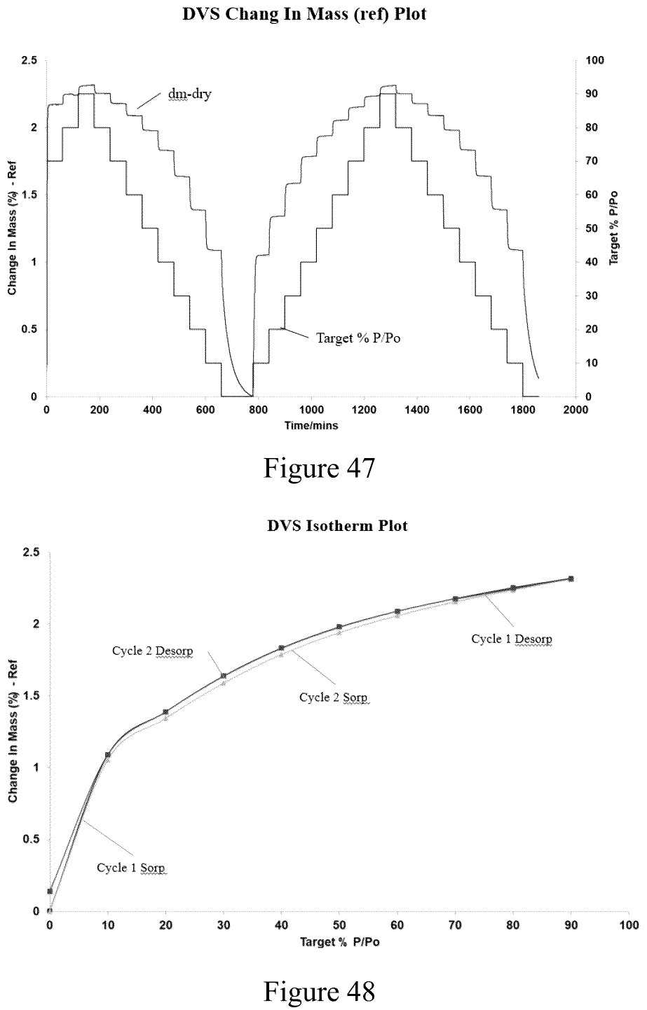

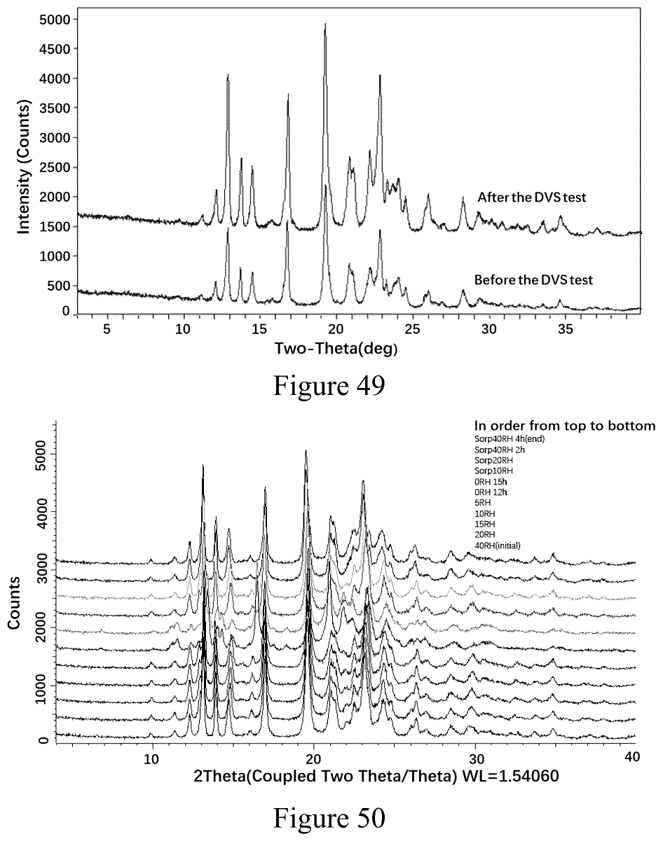

The running procedure of the test is as shown in FIG. 47, the DVS curve is as shown in FIG. 48, and the XRPD superposition pattern before and after the DVS test is as shown in FIG. 49.

The results showed that the adsorption and desorption curves of crystal form III almost completely overlapped, indicating that crystal form III did not form a hydrate or formed a non-stoichiometric hydrate during the moisture absorption process. Combined with the low dehydration temperature in the aforementioned TGA pattern (FIG. 11), crystal form III is a non-stoichiometric hydrate.

The absorption curve in FIG. 48 showed that there was a sudden change in weight in the range of 0 to 10% RH, during which the sample quickly absorbed water, and then the weight gain slowed down. It is inferred that the dehydrated crystal form III can easily return to the water-bearing state under low humidity conditions, and crystal form III is slightly hygroscopic with an adsorbed water content of about 1.2% in the range of 10 to 80% RH.

### 15.2 Stability Investigation Experiment II of Crystal Form III

Based on the test results of Example 13, the stability of crystal form III during a cycle of moisture desorption-absorption of 40% RH-0% RH-40% RH at 25° C. was investigated in order to confirm the control strategy of crystal form III.

The results showed (see Table G): during the cycle of moisture desorption-absorption of 40% RH-0% RH-40% RH at 25° C., it was found that crystal form III was completely dehydrated at 0% RH and transformed into a new crystal form, anhydrous crystal form X, which could be reversibly transformed into crystal form III. Combined with the completely consistent characteristics of the DVS adsorption-desorption curve of crystal form III, it is determined that crystal form III is a non-stoichiometric hydrate.

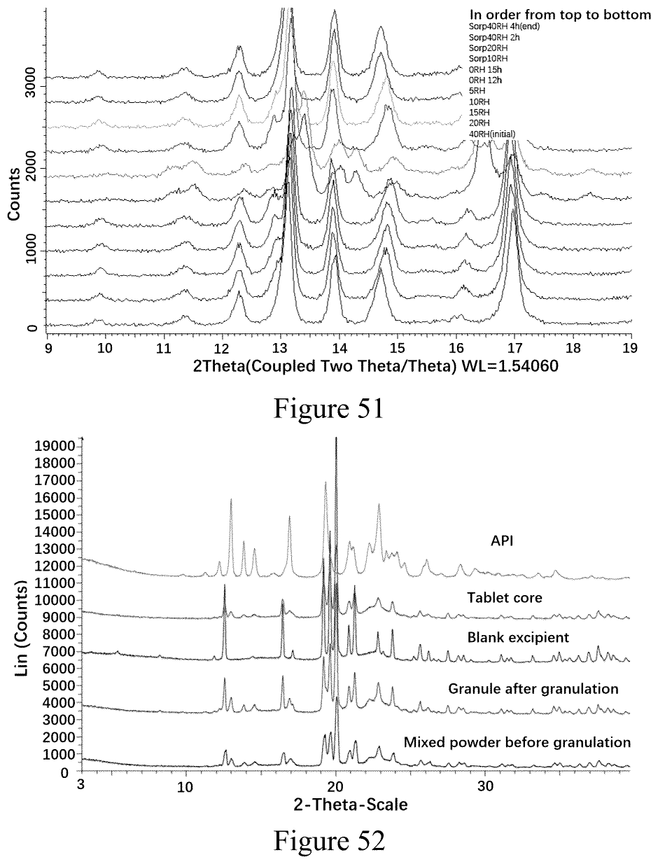

In the range of 50% RH to 200 RH, there is a slight difference at the position of about 12.9° (2θ) under different humidity conditions. However, the crystal form patterns (see FIG. 50—in situ test results of variable humidity XRPD of crystal form III, and FIG. 51—in situ test results of variable humidity XRPD of crystal form III (partial enlargement)) showed the characteristic diffraction peaks of crystal form III, and the sample was completely transformed into crystal form III at 400 RH. Combined with the water content at 40a RH shown in the DVS pattern and the TGA results of crystal form III, crystal form III is stable with the water content controlled above 1.6%.

TABLE G
Summary of VH-XRPD results of crystal form III

| | Humidity change | |
|---|---|---|
| | conditions (humidity, | |
| Procedure | retention time) | XRPD results |
| 1 (start) | 40% RH, 60 minutes | Initial crystal form (crystal form |
| | | III) |
| 2 | 20% RH, 120 minutes | Crystal form III + diffraction |
| | | peak at about 12.9° (2θ) |
| 3 | 15% RH, 120 minutes | Crystal form III + diffraction |
| | | peak at about 12.9° (2θ) |
| 4 | 10% RH, 120 minutes | Crystal form III + diffraction |
| | | peak at about 12.9° (2θ) |
| 5 | 5% RH, 120 minutes | Crystal form III + diffraction |
| | | peak at about 12.9° (2θ) |
| 6 | 0% RH, 720 minutes | Anhydrous crystal form X |
| 7 | 0% RH, 900 minutes | Anhydrous crystal form X |
| 8 | 10% RH, 120 minutes | Crystal form III + diffraction |
| | | peak at about 12.9° (2θ) |
| 9 | 20% RH, 120 minutes | Crystal form III + diffraction |
| | | peak at about 12.9° (2θ) |
| 10 | 40% RH, 120 minutes | Crystal form III |
| 11 (end) | 40% RH, 240 minutes | Crystal form III |

Note:
This crystal form is defined as a new crystal form X as it has a diffraction peak at 12.9°. However, this crystal form cannot be obtained and accurately characterized due to the extreme conditions.

### Example 16: Pharmacokinetic Analysis of Different Crystal Forms in Animals

The present disclosure preferred crystal forms I, III, and V of the compound in the above examples for pharmacokinetic experiments in Beagles, and the relevant experiments are as follows:

Animal: 9 Beagles, male, divided into three groups with 3 in each group, weight ranging from 10 to 13 kg (source & certificate number: Beijing Marshall Biotechnology Co., Ltd., No. 1103182011000161)

Administration mode and dosage: PO: 10 mg/kg

Preparation: 0.5% CMC-Na was used as the solvent, and 400 mg of each of the three different crystal forms of compound A were weighed and added to the corresponding 79 mL of solvent, respectively. The mixture was subjected to ultrasonication for 20 minutes, added with a stirrer, and mixed thoroughly for 3 hours (the storage period from compound preparation to animal administration at room temperature was less than 6 hours). After the drug solution became a homogeneous and fine suspension, which was observed by the naked eye, the solvent was gradually added until the specified volume reached the target concentration. The preparation was stirred for 10 minutes at room temperature before administration, and stirred continuously during administration.

Time points for sampling: the time points for blood sampling in the PO group were 0.167, 0.5, 1, 2, 4, 6, 8, and 24 hours.

Animals in the PO group were fasted overnight except for free access to water before administration, and resumed food supply 4 hours after administration.

### Sample Treatment:

After Beagles in the two groups were administered, blood was collected from Beagles by jugular vein puncture according to the time points for sampling. About 1.0 mL of blood was collected at each time point and transferred to an anticoagulant EP tube (containing 10 μL of EDTA-K2, 375 mg/mL). The tube was gently inverted three times, stored in an ice box (no more than 30 minutes), and centrifuged at 3200 g for 10 minutes at 4° C. The plasma was transferred to an ultra-low temperature refrigerator and stored until detection.

Sample pretreatment process: 40 μL of sample was taken, and 160 μL of acetonitrile solution containing 0.1% FA and 200 ng/mL of mixed standard solution was added to precipitate the protein. The mixture was vortexed and mixed well, and the sample was centrifuged in a 4° C. centrifuge at 13000 pm for 10 minutes. 100 μL of the supernatant was transferred to another 96 deep well plate, then 100 μL of methanol:water (1:3, v:v) solution containing 0.1% FA was added thereto, and the mixture was shaken and mixed for 10 minutes.

### 3. Sample Detection:

### (1) Mass Spectrometry Conditions

### 1. Mass Spectrometry Parameters

Ion source: ion electrospray (ESI)
Ionization mode: positive ion mode (Positive)
Detection mode (mode): multiple reaction monitoring (MRM)
Ion spray voltage: 5500 V
Ion spray temperature (turbo ion spray temp): 550° C.
Curtain gas type: nitrogen setting: 35
Collision cell gas type (CAD gas type): nitrogen setting: medium
Nebulizing gas type (gas 1): nitrogen setting: 55.00
Auxiliary gas type (gas 2): nitrogen setting: 55.00

### (2) Liquid Phase Conditions

Chromatographic column: poroshell 120 EC-C18 (4.6×50 mm, 2.7 μm)
Mobile phase A: 0.1% formic acid aqueous solution
Mobile phase B: a solution of 0.1% formic acid in methanol
Auto sampler wash solution (rinse port wash solution): 50% methanol
Column temperature: 40° C.
Flow rate: 0.55 mL/min
Auto sampler temperature (sample tray temp): 10° C.
Injection volume: 3 μL
Lift of presser foot (needle stroke): 49 mm
Auto sampler rinse setting (rinse pump setting): rinse port only
Auto sampler rinse mode (rinse mode): before aspiration
Auto sampler rinse volume for needle (rinse volume): 500 μL
Dip time when rinsing the injection needle (rinse dip time): 2 seconds

### Elution Gradient:

| Time (min) | Module | Function | Value (%) |
|---|---|---|---|
| 0.01 | Pumps | Pump B Conc. | 30 |
| 0.20 | Pumps | Pump B Conc. | 30 |
| 1.20 | Pumps | Pump B Conc. | 98 |
| 2.40 | Pumps | Pump B Conc. | 98 |
| 2.41 | Pumps | Pump B Conc. | 30 |
| 3.00 | System | Stop | |
| | Controller | | |

### Data Analysis:

The data would be analyzed using WinNonlin (version 5.2.1 Pharsight, Mountain View, CA) through a non-compartmental model to obtain PK parameters (selecting Cₘₐₓ, Tₘₐₓ, AUCₗₐₛₜ, T_{1/2}, and other parameters according to different routes of administration). The results are shown in the table below.

| Crystal | Animal | Route of | | Dose | Cₘₐₓ | Tₘₐₓ | T_{1/2} | AUCₗₐₛₜ |
|---|---|---|---|---|---|---|---|---|
| form | species | administration | Analyte | (mg/kg) | (ng/mL) | (hr) | (hr) | (ng/mL * hr) |
| I | Beagle, | PO | Plasma | 10 | 991 ± 800 | 2.00 ± 0.00 | 11.9 ± 3.65 | 7926 ± 6235 |
| | male | | concentration | | | | | |
| V | Beagle, | PO | Plasma | 10 | 170 ± 99.4 | 4.67 ± 0.67 | 44.4 ± 28.9 | 2637 ± 1750 |
| | male | | concentration | | | | | |
| III | Beagle, | PO | Plasma | 10 | 1159 ± 160 | 2.00 ± 0.00 | 12.9 ± 1.06 | 14454 ± 3182 |
| | male | | concentration | | | | | |

### Example 17: Formulation and Process of the Preparation of the Present Disclosure

### Example 17.1: Experimental Raw Materials

TABLE H-1
Experimental raw materials

| No. | Material | Function | Supplier |
|---|---|---|---|
| 1 | Compound of | Active | — |
| | formula A | ingredient | |
| 2 | Lactose monohydrate | Diluent | Molkerei MEGGLE |
| | FLOWLAC ®100 | | Wasserburg GmbH & |
| | | | Co. KG |
| 3 | Microcrystalline | | DuPont Nutrition |
| | cellulose 102 | | USA, Inc. |
| 4 | Croscarmellose | Disintegrant | DuPont Nutrition |
| | sodium | | USA, Inc. |
| 5 | Hydroxypropyl | Adhesive | Ashland |
| | cellulose EXF | | |
| 6 | Colloidal silica | Glidant | Evonik Specialty |
| | 200 | | Chemicals (Shanghai) |
| | | | Co., Ltd. |
| 7 | Magnesium stearate | Lubricant | Peter Greven |
| | LIGAMED MF-2-V | | Germany |
| 8 | Film coating | Coating | Shanghai Colorcon |
| | premix (gastric- | material | Coating Technology |
| | soluble) - Opadry ® | | Co., Ltd. |
| | 85F18422-CN, US | | |
| 9 | 45 mL oral solid | Packaging | Sanner Pharmaceutical |
| | pharmaceutical | material | Packaging Materials |
| | high-density | | Co., Ltd. |
| | polyethylene bottle | | |
| 10 | 75 mL oral solid | | |
| | pharmaceutical | | |
| | high-density | | |
| | polyethylene bottle | | |
| 11 | Oral solid | | |
| | pharmaceutical | | |
| | child resistant cap | | |

### 17.2 Prescriptions for 25 mg and 100 mg Coated Tablets as Shown in Table H-2

TABLE H-2
Preparation prescription

| | | | |
|---|---|---|---|
| | | Specification: | Specification: |
| | | 25 mg, | 100 mg, |
| | | calculated | calculated |
| | | as active | as active |
| | | ingredient | ingredient |
| Composition | % w/w | mg/tablet | mg/tablet |

Internal addition

| | | | |
|---|---|---|---|
| Compound of formula A*⁴ | 25.0 | 25.0 | 100.0 |
| Colloidal silica 200 | 1.0 | 1.0 | 4.0 |
| Lactose monohydrate | 49.6 | 49.6 | 198.4 |
| FLOWLAC ®100 | | | |
| Microcrystalline | 17.4 | 17.4 | 69.6 |
| cellulose 102 | | | |
| Hydroxypropyl | 3.0 | 3.0 | 12.0 |
| cellulose EXF | | | |
| Croscarmellose | 3.0 | 3.0 | 12.0 |
| sodium | | | |
| Purified water*¹ | N/A | N/A | N/A |

External addition

| | | | |
|---|---|---|---|
| Magnesium stearate | 1.0 | 1.0 | 4.0 |
| Total tablet cores | 100.0 | 100.0 | 400.0 |

Coated tablet

| | | | |
|---|---|---|---|
| Film coating premix | 2.0 to 4.0 | 2.0 to 4.0 | 8.0 to 16.0 |
| (gastric-soluble) - | | | |
| Opadry ® 85F18422- | | | |
| CN, US*² | | | |
| Purified water*³ | N/A | N/A | N/A |
| Total coated tablets | N/A | 102.0 to 104.0 | 408.0 to 416.0 |

Remarks:
*¹The concentration of the adhesive is 9%. Purified water is removed during the drying process as a solvent in the wet granulation process, and is not included in the material balance. The actual amount of purified water can be adjusted appropriately according to the actual situation of wet granulation.
*²The coating weight gain range is controlled at 2.0 to 4.0% of the average tablet weight, and the calculation formula is (average total weight of 20 coated tablets − total weight of 20 tablet cores)/total weight of 20 tablet cores * 100%.
*³The solid content of the coating suspension is 15%. Purified water is removed during the coating process as a dispersing medium for the coating powder, and is not included in the material balance.
*⁴The compound of formula A is crystal form III.

### 17.3 Preparation Process of 25 mg Coated Tablets and 100 mg Coated Tablets

### (1) Weighing

Equipment: balance
Step: Raw materials were weighed for a later use.

### (2) Pre-Processing

Equipment: 60 mesh sieve, 40 mesh sieve
Step: The compound of formula A was mixed with colloidal silica 200 in a low-density polyethylene bag for 1 minute, and the mixture was sieved through a 60 mesh sieve, the 60 mesh sieve was then rinsed with microcrystalline cellulose 102. All mixtures were collected and placed in the same low-density polyethylene bag as mixture 1.

Lactose monohydrate was mixed with croscarmellose sodium in a low-density polyethylene bag for 1 minute, and the mixture was sieved through a 40 mesh sieve as mixture 2.

Magnesium stearate was sieved once through the same 40 mesh sieve, and placed in another pharmaceutical low-density polyethylene bag.

### (3) Adhesive Preparation

Equipment: overhead electronic mixer, pneumatic mixer
Step: Purified water was weighed, hydroxypropyl cellulose EXF was slowly added to the purified water with stirring until completely dissolved, and an adhesive solution with a concentration of 9% was prepared.

### (4) Wet Granulation

Equipment: wet granulator
Step 1: Mixing: About ½ of mixture 2 and mixture 1 were placed in a wet granulation pot, the low-density polyethylene bag containing mixture 1 was rinsed with the remaining mixture 2, and added to the wet granulation pot. The stirring speed was set at 250.0 rpm, the cutting speed was set at 450.0 rpm, and the mixing time was set at 5 minutes. After the mixing was completed, the weight loss on drying of the material was measured, and the moisture was detected as needed.
Step 2: Spraying: The stirring speed was set at 250.0 rpm, the cutting speed was set at 1500.0 rpm, and the prepared adhesive was sprayed into the wet granulation pot. The wet granulation spraying speed was adjusted to about 200 to 347 g/min.
Step 3: Water addition: The granulation situation was observed after the adhesive was added, and if additional water was needed, an appropriate amount of water was added.
Step 4: Granulation: The stirring speed was set at 250.0 rpm, the cutting speed was set at 1500.0 rpm, and the granulation time was set at 60 seconds. Wet granules were obtained.
IPC: Mixed powder: weight loss on drying, moisture detection as needed.

### (5) Wet Pelletization

Equipment: pelletizer, 6×6 mm sieve mesh
Step: The wet granules were pelletized in a pelletizer with a speed of 1000.0 rpm, and sieved through a mesh sieve with an aperture of 6×6 mm.

### (6) Drying

Equipment: fluidized bed
Step 1: Preheating: The equipment parameters were set as follows: inlet air temperature: 50 to 60° C., inlet air volume: 20 to 150 m³/h. When the outlet air temperature reached about 35° C. or more, the wet particles were added for drying.
Step 2: Drying: inlet air temperature: 50 to 60° C., inlet air volume: 20 to 150 m³/h, filter bag shaking time: 1±0.8 seconds, filter bag shaking frequency: 8±5 seconds, back blowing pressure: 200 to 350 kPa, the equipment parameters were recorded every 10 minutes. When the product temperature reached about 30° C., a sample of 2 to 3 g was taken to measure the weight loss on drying of the material. When the weight loss on drying of the material was between 1.0% and 2.5%, the inlet air temperature was turned off to stop drying, the value of weight loss on drying was recorded, and the moisture was detected as needed.
IPC: weight loss on drying, moisture detection as needed.

### (7) Dry Pelletization

Equipment: pelletizer, φ1.0 mm round hole sieve mesh
Step: The dry granules were pelletized in a pelletizer with a speed of 1000.0 rpm, and sieved through a φ1.0 mm round hole sieve mesh.

### (8) Lubrication

Equipment: hopper mixer
Step: Some of the dry granules, magnesium stearate, and the remaining dry granules were placed in a 15 L hopper for total mixing, with a mixing speed of 20.0 r/min and a mixing time of 5 minutes.
IPC: mixing uniformity

### (9) Tabletting

Equipment: tablet press, top rotary sieving machine, metal detector
Step: The material was added to the hopper of the tablet press for tabletting.
25 mg specification: 6 mm shallow concave round punch, tablet core target weight per tablet: 100.0 mg, tablet core weight per tablet controlled at ±7.5%, tablet core hardness per tablet controlled at 35 to 90 N;
100 mg specification: 10 mm shallow concave round punch, tablet core target weight per tablet: 400.0 mg, tablet core weight per tablet controlled at ±5.0%, tablet core hardness per tablet controlled at 70 to 130 N;
IPC: tablet weight, hardness, disintegration time, friability, and appearance.

### (10) Coating

Equipment: coating machine, overhead electronic mixer
Step 1: Coating solution preparation: Purified water was weighed, and the stirring was started. The coating powder was slowly added to the purified water at room temperature. After complete addition, the mixture was stirred for another 45 minutes or more to disperse the coating solution uniformly, and a coating solution with a solid content of 15% was obtained.
Step 2: Preheating: The inlet air temperature was set at 50 to 70° C., the inlet air volume was set at 400±100 m³/h, and the coating pot rotation speed was set at 2 to 5 rpm. The two obtained tablet cores were preheated, and coating was started when the outlet air temperature reached about 45° C.
Step 3: Spraying: the inlet air temperature was set at 50 to 70° C., the pot rotation speed was set at 5 to 15 rpm, the inlet air volume was set at 400±100 m³/h, the pump flow rate was set at 4 to 20 mL/min, the atomization pressure was set at 1.5±1.0 bar, and the atomization angle control pressure was set at 2.0±1.0 bar. During the coating process, the pot temperature and atomized coating should be focused, and there should be no tablet sticking phenomenon. Coating weight gain was controlled at 2.0 to 4.0%.
Step 4: Drying and cooling: After the spraying was completed, the inlet air temperature was set at 50 to 70° C., the coating pot rotation speed was adjusted to 2 to 5 rpm, the inlet air volume was set at 400±100 m³/h, and the mixture was dried for 5 minutes. Heating was stopped, and the inlet air volume was set at 400±100 m³/h. The mixture was cooled for at least 5 minutes, then discharged, and placed in a pharmaceutical low-density polyethylene bag.
IPC: coating weight gain

### (11) Packaging and Labeling

Equipment: electromagnetic induction aluminum foil sealing machine, electronic automatic counting machine
Step: 25 mg coated tablets were packed into 45 mL oral solid pharmaceutical high-density polyethylene (HDPE) bottles with a packaging specification of 30 tablets/bottle. A bottle label was put on each bottle.

100 mg coated tablets were packed into 75 mL oral solid pharmaceutical high-density polyethylene (HDPE) bottles with a packaging specification of 30 tablets/bottle. A bottle label was put on each bottle.

### Example 18 Investigation of Preparation Process on Stability of Crystal Form III

In order to investigate the impact of the preparation process of the preparation on the crystal form, samples were taken at different stages during the preparation process of the coated tablets according to Example 17.3 and analyzed. The analysis results are shown in Table I. The summarized results of the XRPD patterns of the products at each time point for sampling are shown in FIG. 52. The results showed that the crystal form of the API remained stable during the wet granulation process and tabletting step.

TABLE I
Statistics of crystal forms in preparation process

| Sampling | Crystal form |
|---|---|
| API | Crystal form III |
| Tablet core | Crystal form III |
| Blank excipient | — |
| Granule after granulation | Crystal form III |
| Mixed powder before granulation | Crystal form III |

### Example 19: Dissolution Detection of Coated Tablets and Investigation of Crystal Changes in AP

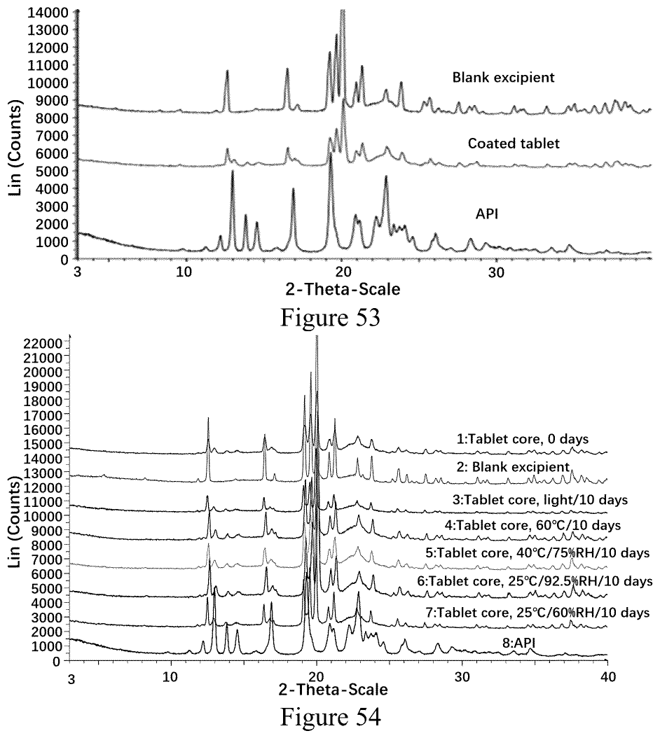

The investigation results are shown in Table J. The XRPD results of the crystal form of the coated tablet, API, and the blank excipient are shown in FIG. 53. From the results shown in Table J and FIG. 53, it can be seen that the 25 mg and 100 mg coated tablets dissolved quickly and eventually dissolved completely, and the crystal form of API did not change after coating.

TABLE J
Dissolution results of coated tablets

| | | | Dissolution (%) | | | | | |
|---|---|---|---|---|---|---|---|---|
| | | | (Paddle method, 50 rpm, | | | | | |
| | | | 0.1N HCL, 900 mL, n = 6) | | | | | |
| Specifi- | | | 5 | 15 | 30 | 45 | 60 | 75 min |
| cation | | Item | min | min | min | min | min | (ultimate) |
| 25 | mg | Average (%) | 93 | 98 | 100 | 100 | 100 | 100 |
| | | RSD (%) | 1.9 | 1.7 | 1.6 | 1.4 | 1.2 | 1.2 |
| 100 | mg | Average (%) | 87 | 94 | 97 | 98 | 99 | 102 |
| | | RSD (%) | 5.6 | 2.0 | 1.7 | 1.4 | 1.5 | 1.1 |

### Example 20: Investigation of Tablet Stability

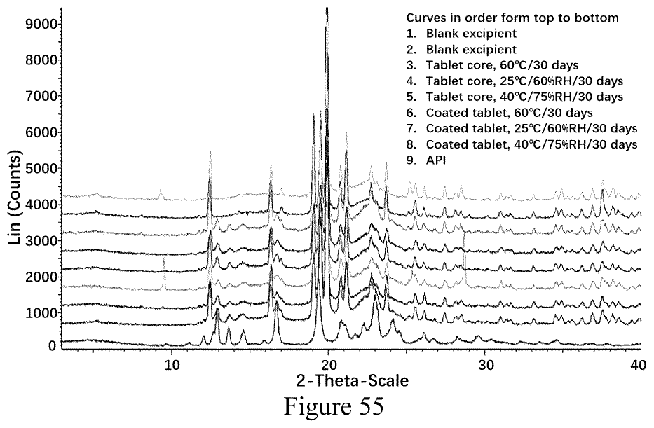

25 mg and 100 mg tablet cores and coated tablets were investigated for their stability. The results of related substances are shown in Table K-1 and Table K-2. The results of dissolution detection are shown in Table K-3 and Table K-4. The results of crystal form stability are shown in FIG. 54 and FIG. 55.

TABLE K-1
Stability of related substances in tablet cores
Related substance %
25 mg tablet core

| | RRT | | | | | | | RRT | |
|---|---|---|---|---|---|---|---|---|---|
| | 0.64/0.66/ | RRT | RRT | RRT | RRT | RRT | RRT | 1.36/1.39/ | Total |
| Condition | 0.67 | 0.95 | 1.03 | 1.08 | 1.09/1.10 | 1.15/1.17 | 1.36 | 1.43 | impurities |
| 0 days | 0.09 | 0.48 | 0.06 | 0.94 | 0.30 | ND | ND | 0.57 | 2.44 |
| 60° C./open/10 | 0.09 | 0.48 | 0.06 | 0.93 | 0.29 | ND | ND | 0.57 | 2.42 |
| days | | | | | | | | | |
| 25° C./92.5% | 0.09 | 0.48 | 0.06 | 0.93 | 0.29 | ND | 0.05 | 0.57 | 2.47 |
| RH/open/10 | | | | | | | | | |
| days | | | | | | | | | |
| 40° C./75% | 0.09 | 0.49 | 0.06 | 0.94 | 0.29 | ND | ND | 0.57 | 2.44 |
| RH/closed/10 | | | | | | | | | |
| days | | | | | | | | | |
| Light/open/10 | 0.07 | 0.58 | 0.07 | 0.92 | 0.31 | 1.05 | ND | 0.56 | 3.56 |
| days | | | | | | | | | |
| Light/open/20 | 0.07 | 0.60 | 0.06 | 0.85 | 0.19 | 1.41 | ND | 0.57 | 3.75 |
| days | | | | | | | | | |
| Light/closed/10 | 0.08 | 0.51 | 0.05 | 0.89 | 0.23 | ND | ND | 0.57 | 2.33 |
| days | | | | | | | | | |

Remarks: Under the conditions of 25° C./92.5% RH/open/30 days, the tablet core was mildewed, so it was not detected.

TABLE K-2
Stability of related substances in coated tablets

| | RRT | | | | RRT | | | RRT | |
|---|---|---|---|---|---|---|---|---|---|
| | 0.63/0.66/ | RRT | RRT | RRT | 1.08/1.09/ | RRT | RRT | 1.31/1.36/ | Total |
| Condition | 0.67/0.69 | 0.95/0.96 | 1.03 | 1.07/1.08 | 1.10 | 1.15/1.17 | 1.36 | 1.39/1.46 | impurities |

Related substance %
25 mg coated tablet

| | | | | | | | | | |
|---|---|---|---|---|---|---|---|---|---|
| 0 days | 0.08 | 0.48 | 0.07 | 0.92 | 0.31 | ND | ND | 0.58 | 2.44 |
| 60° C./open/ | 0.09 | 0.49 | 0.06 | 0.93 | 0.29 | ND | ND | 0.57 | 2.43 |
| 10 days | | | | | | | | | |
| 60° C./open/ | 0.09 | 0.47 | 0.06 | 1.00 | 0.39 | ND | ND | 0.58 | 2.59 |
| 30 days | | | | | | | | | |
| 25° C./92.5% | 0.09 | 0.49 | 0.06 | 0.94 | 0.29 | ND | ND | 0.57 | 2.44 |
| RH/open/ | | | | | | | | | |
| 10 days | | | | | | | | | |
| 40° C./75% | 0.09 | 0.49 | 0.06 | 0.94 | 0.29 | ND | ND | 0.57 | 2.44 |
| RH/closed/ | | | | | | | | | |
| 10 days | | | | | | | | | |
| 40° C./75% | 0.09 | 0.46 | 0.05 | 1.00 | 0.38 | ND | ND | 0.58 | 2.56 |
| RH/closed/ | | | | | | | | | |
| 30 days | | | | | | | | | |
| Light/open/ | 0.08 | 0.58 | 0.06 | 0.93 | 0.31 | 0.05 | ND | 0.57 | 2.58 |
| 10 days | | | | | | | | | |
| Light/open/ | 0.07 | 0.58 | 0.05 | 0.87 | 0.21 | 0.08 | ND | 0.56 | 2.42 |
| 20 days | | | | | | | | | |
| Light/closed/ | 0.09 | 0.50 | 0.05 | 0.89 | 0.25 | ND | ND | 0.58 | 2.36 |
| 10 days | | | | | | | | | |

Related substance %
100 mg coated tablet

| | | | | | | | | | |
|---|---|---|---|---|---|---|---|---|---|
| Room | 0.09 | 0.47 | 0.06 | 0.98 | 0.34 | ND | ND | 0.58 | 2.52 |
| temperature/ | | | | | | | | | |
| 30 days | | | | | | | | | |
| 60° C./open/ | 0.08 | 0.47 | 0.06 | 0.97 | 0.34 | ND | ND | 0.58 | 2.50 |
| 30 days | | | | | | | | | |
| 40° C./75% | 0.09 | 0.48 | 0.06 | 0.98 | 0.36 | ND | ND | 0.58 | 2.55 |
| RH/closed/ | | | | | | | | | |
| 30 days | | | | | | | | | |

Remarks: Under the conditions of 25° C./92.5% RH/open/30 days, the coated tablets were mildewed, so they were not detected.

It can be seen from the above results that for 25 mg tablet core, there was no significant change in the related substances after being placed under the conditions of 60° C./open, 25° C./92.5% RH/open, 40° C./75% RH/closed, and light/closed for 10 days, while there was a significant increase in the related substances after being placed under the conditions of light/open for 10 days and 20 days, compared with the result that there was no significant change in the related substances of coated tablets, indicating that the coating can effectively avoid the impact of light on the product.

For 25 mg and 100 mg coated tablets, there was no significant change in the related substances after being placed under the conditions of 60° C./open and 40° C./75% RH/closed for 30 days; there was no significant change in the related substances after being placed under the conditions of light/open for 20 days and light/closed for 10 days; and there was no significant change in the related substances after being placed under the conditions of 25° C./92.5% RH/open for 10 days (mildew occurred after being placed for 30 days, not detected). The above results of the stability of the related substances indicate that the coated tablet has good stability.

TABLE K-3
Dissolution stability of 100 mg tablet core

| | | | Dissolution (%) | | | | | |
|---|---|---|---|---|---|---|---|---|
| | | | (Paddle method, 50 rpm, 0.1N HCL, 900 mL, | | | | | |
| | | | 0 days n = 6, stability n = 3) | | | | | |
| Batch | | | | | | | | 75 min |
| number | Condition | Item | 5 min | 15 min | 30 min | 45 min | 60 min | (ultimate) |
| 100 mg | 0 days | Average (%) | 90 | 95 | 97 | 98 | 98 | 100 |
| tablet core | | RSD (%) | 2.1 | 2.5 | 2.6 | 2.1 | 2.0 | 1.0 |
| | 60° C./open/10 days | Average (%) | 93 | 97 | 98 | 99 | 99 | 101 |
| | | RSD (%) | 0.4 | 0.7 | 0.4 | 0.1 | 0.4 | 0.5 |
| | 40° C./75% | Average (%) | 93 | 96 | 97 | 98 | 99 | 101 |
| | RH/closed/10 days | RSD (%) | 1.7 | 1.6 | 1.8 | 1.7 | 1.7 | 1.3 |
| | 25° C./92.5% | Average (%) | 88 | 94 | 96 | 97 | 98 | 99 |
| | RH/open/10 days | RSD (%) | 1.5 | 1.0 | 0.6 | 0.7 | 0.8 | 0.8 |
| | Light/open/10 days | Average (%) | 88 | 92 | 94 | 95 | 96 | 99 |
| | | RSD (%) | 2.7 | 2.2 | 2.1 | 2.0 | 2.0 | 1.5 |

TABLE K-4
Dissolution stability of 25 mg and 100 mg coated tablets

| | | | Dissolution (%) | | | | | |
|---|---|---|---|---|---|---|---|---|
| | | | (Paddle method, 50 rpm, 0.1N HCL, 900 mL, | | | | | |
| | | | 0 days n = 6, stability n = 3) | | | | | |
| Batch | | | | | | | | 75 min |
| number | Condition | Item | 5 min | 15 min | 30 min | 45 min | 60 min | (ultimate) |
| 100 mg | 0 days | Average (%) | 81 | 90 | 92 | 94 | 95 | 99 |
| coated | | RSD (%) | 5.9 | 3.6 | 3.0 | 2.9 | 2.6 | 1.2 |
| tablet | 60° C./open/10 days | Average (%) | 88 | 94 | 96 | 97 | 98 | 101 |
| | | RSD (%) | 3.3 | 1.0 | 0.7 | 0.5 | 0.7 | 0.4 |
| | 60° C./open/30 days | Average (%) | 88 | 95 | 97 | 98 | 98 | 101 |
| | | RSD (%) | 4.9 | 2.6 | 2.2 | 1.9 | 1.8 | 1.2 |
| | 40° C./75% | Average (%) | 85 | 93 | 96 | 97 | 98 | 102 |
| | RH/closed/10 days | RSD (%) | 1.1 | 0.6 | 0.1 | 0.2 | 0.4 | 0.8 |
| | 40° C./75% | Average (%) | 90 | 94 | 96 | 97 | 97 | 101 |
| | RH/closed/30 days | RSD (%) | 1.1 | 0.5 | 0.5 | 0.6 | 0.5 | 0.8 |
| | 25° C./92.5% | Average (%) | 85 | 91 | 93 | 95 | 95 | 99 |
| | RH/open/10 days | RSD (%) | 2.8 | 1.7 | 0.9 | 0.9 | 0.8 | 0.2 |
| | Light/open/10 days | Average (%) | 85 | 92 | 94 | 95 | 96 | 99 |
| | | RSD (%) | 2.7 | 1.1 | 0.7 | 0.7 | 0.7 | 0.7 |
| 25 mg | 0 days | Average (%) | 92 | 98 | 99 | 100 | 100 | 100 |
| coated | | RSD (%) | 3.2 | 1.1 | 0.9 | 0.9 | 1.0 | 1.1 |
| tablet | 60° C./open/30 days | Average (%) | 54 | 99 | 101 | 101 | 101 | 101 |
| | | RSD (%) | 32.8 | 1.2 | 1.4 | 1.4 | 1.4 | 1.6 |
| | 40° C./75% | Average (%) | 89 | 96 | 98 | 99 | 99 | 100 |
| | RH/closed/30 days | RSD (%) | 1.9 | 1.7 | 1.7 | 1.6 | 1.5 | 1.2 |

From the above results, it can be seen that there was no significant change in the dissolution rate of 100 mg tablet core after being placed under the conditions of 60° C./open, 40° C./75% RH/closed, 25° C./92.5% RH/open, and light/open for 10 days. There was no significant change in the dissolution rate of 25 mg and 100 mg coated tablets after being placed under the conditions of 60° C./open and 40° C./75% RH/closed for 30 days; and there was no significant change in the dissolution rate of 100 mg coated tablet after being placed under the conditions of 25° C./92.5% RH/open and light/open for 10 days. The above data indicate that the product has good dissolution stability.

From the the results of the crystal stability investigation of the 100 mg tablet core and coated tablet as shown in FIG. 54 and FIG. 55, it can be seen that there was no change in the crystal forms of the tablet core and coated tablet after being placed under the conditions of 60° C./open, 40° C./75% RH/closed, and 25° C./60% RH/open for 30 days, indicating that the product has good physical stability.

It has been experimentally demonstrated that the pharmaceutical compositions and preparations of the present disclosure have good safety and/or stability, as well as high P2X3 antagonistic activity with less impact on the taste.

The embodiments of the present disclosure have been described above. However, the present disclosure is not limited to the above embodiments. Any modifications, equivalent substitutions, improvements, etc. made within the spirit and principles of the present disclosure shall be included in the scope of protection of the present disclosure.

1. A pharmaceutical composition, which is in a solid form, and comprises an active ingredient and a pharmaceutically acceptable excipient; the active ingredient comprises a compound of formula A: 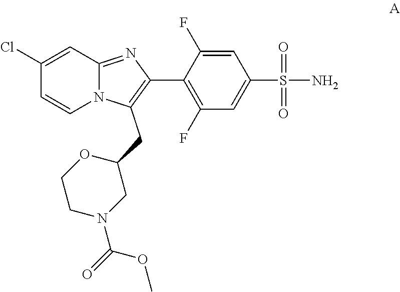 [SMILES: COC(=O)N1CCO[C@@H](Cc2c(-c3c(F)cc(S(N)(=O)=O)cc3F)nc3cc(Cl)ccn23)C1]
the excipient is selected from one, two or more of the following excipients comprising, but not limited to, a diluent, a disintegrant, an adhesive, a glidant, and a lubricant; the compound of formula A is selected from one, two or more of a crystal form I, a crystal form III, and a crystal form V;
the crystal form I has characteristic peaks at 8.56°±0.20°, 12.48°±0.20°, and 22.13°±0.20° in an X-ray powder diffraction expressed at a 2θ angle using Cu-Kα radiation;
the crystal form III has characteristic peaks at 12.91°±0.20°, 16.77°±0.20°, 19.27°±0.20°, and 22.80°±0.20° in an X-ray powder diffraction expressed at a 2θ angle using Cu-Kα radiation;
the crystal form V has characteristic peaks at 8.38°±0.20°, 9.15°±0.20°, 13.52°±0.20°, and 18.44±0.20° in an X-ray powder diffraction expressed at a 2θ angle using Cu-Kα radiation.

2. The pharmaceutical composition according to claim 1, wherein the pharmaceutical composition satisfies one or more of the following conditions:
(1) the compound of formula A is selected from the crystal form III;
(2) the diluent is selected from one, two or more of the following substances: lactose, microcrystalline cellulose, sucrose, glucose, mannitol, sorbitol, calcium sulfate, calcium gluconate, calcium hydrogen phosphate, calcium phosphate, calcium carbonate, calcium bicarbonate, starch, carboxymethyl starch, and pregelatinized starch;
(3) the disintegrant is selected from one, two or more of the following substances: croscarmellose sodium, pregelatinized starch, microcrystalline cellulose, alginic acid, lignocellulose, sodium carboxymethyl starch, guar gum, and cross-linked polyvinylpyrrolidone;
(4) the adhesive is selected from one, two or more of the following substances: hydroxypropyl cellulose, gelatin, dextrin, maltodextrin, sucrose, gum arabic, polyvinylpyrrolidone, methyl cellulose, carboxymethyl cellulose, ethyl cellulose, polyvinyl alcohol, polyethylene glycol, and hydroxypropyl methyl cellulose;
(5) the glidant is selected from one, two or more of the following substances: colloidal silica, silica, talc, calcium silicate, magnesium silicate, and calcium hydrogen phosphate;
(6) the lubricant is selected from one, two or more of the following substances: magnesium stearate, calcium stearate, zinc stearate, talc, glycerol monostearate, polyethylene glycol, sodium benzoate, adipic acid, fumaric acid, boric acid, sodium chloride, sodium oleate, triacetin, polyoxyethylene monostearate, sucrose monolaurate, sodium chloride, sodium lauryl sulfate, and magnesium lauryl sulfate;
(7) the pharmaceutical composition comprises 10 to 40 parts by weight of the compound of formula A;
(8) the pharmaceutical composition comprises 50 to 80 parts by weight of the diluent;
(9) the pharmaceutical composition comprises 0.5 to 6 parts by weight of the disintegrant;
(10) the pharmaceutical composition comprises 0.5 to 6 parts by weight of the adhesive;
(11) the pharmaceutical composition comprises 0.1 to 3 parts by weight of the glidant;
(12) the pharmaceutical composition comprises 0.1 to 3 parts by weight of the lubricant;
(13) the sum of parts by weight of each component in the pharmaceutical composition is 100 parts;
(14) the pharmaceutical composition is in a powdered solid form.

3. The pharmaceutical composition according to claim 1, wherein the pharmaceutical composition satisfies one or more of the following conditions:
(1) the crystal form III has characteristic peaks at 12.91°±0.20°, 16.77°±0.20°, 19.27°±0.20°, 22.80°±0.20°, 13.75°±0.20°, 14.46°±0.20°, and 20.86°±0.20° in an X-ray powder diffraction expressed at a 2θ angle using Cu-Kα radiation; preferably, the crystal form III has an XRPD pattern substantially as shown in FIG. 10;
(2) the compound of formula A has a particle size of 1 to 40 μm;
(3) the compound of formula A has a loose density (bulk density) of 0.2 to 0.3 g/mL.

4. The pharmaceutical composition according to claim 1, wherein the pharmaceutical composition satisfies one or more of the following conditions:
(1) the diluent comprises a first diluent and a second diluent, and the first diluent and the second diluent are different and independently selected from one of the following substances: lactose, microcrystalline cellulose, sucrose, glucose, mannitol, sorbitol, calcium sulfate, calcium gluconate, calcium hydrogen phosphate, calcium phosphate, calcium carbonate, calcium bicarbonate, starch, carboxymethyl starch, and pregelatinized starch; preferably, the first diluent is microcrystalline cellulose, and the second diluent is lactose monohydrate;
(2) the excipient further comprise a corrigent;
(3) the diluent is lactose monohydrate and microcrystalline cellulose, the disintegrant is croscarmellose sodium, the adhesive is hydroxypropyl cellulose, the glidant is colloidal silica, and the lubricant is magnesium stearate.

5. The pharmaceutical composition according to claim 1, wherein
the pharmaceutical composition comprises the following components: the compound of formula A, lactose monohydrate, microcrystalline cellulose, croscarmellose sodium, hydroxypropyl cellulose, colloidal silica, and magnesium stearate, preferably, the pharmaceutical composition comprises the following components by weight: 10 to 40 parts of the compound of formula A, a total of 50 to 80 parts of lactose monohydrate and microcrystalline cellulose, 0.5 to 6 parts of croscarmellose sodium, 0.5 to 6 parts of hydroxypropyl cellulose, 0.1 to 3 parts of colloidal silica, and 0.1 to 3 parts of magnesium stearate.

6. A pharmaceutical preparation, comprising the pharmaceutical composition according to claim 1.

7. A method for preparing the pharmaceutical composition according to claim 1, comprising mixing the components comprised therein; preferably, sieving a prescribed amount of the compound of formula A, the glidant and the first diluent first, and then mixing with other components.

8. (canceled)

9. A method for storing the pharmaceutical composition according to claim 1, comprising storing the pharmaceutical composition in the dark; further, storage conditions further comprise dry storage.

10. A use of a pharmaceutical composition in the manufacture of the pharmaceutical preparation according to claim 6;
the pharmaceutical composition is in a solid form, and comprises an active ingredient and a pharmaceutically acceptable excipient; the active ingredient comprises a compound of formula A: 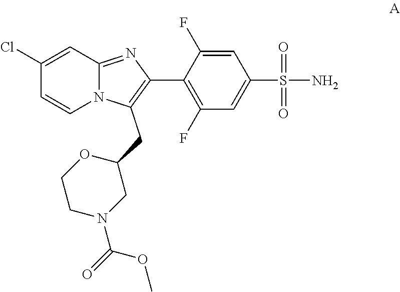 [SMILES: COC(=O)N1CCO[C@@H](Cc2c(-c3c(F)cc(S(N)(=O)=O)cc3F)nc3cc(Cl)ccn23)C1]
the excipient is selected from one, two or more of the following excipients comprising, but not limited to, a diluent, a disintegrant, an adhesive, a glidant, and a lubricant; the compound of formula A is selected from one, two or more of a crystal form I, a crystal form III, and a crystal form V;
the crystal form I has characteristic peaks at 8.56°±0.20°, 12.48°±0.20°, and 22.13°±0.20° in an X-ray powder diffraction expressed at a 2θ angle using Cu-Kα radiation;
the crystal form III has characteristic peaks at 12.91°±0.20°, 16.77°±0.20°, 19.27°±0.20°, and 22.80°±0.20° in an X-ray powder diffraction expressed at a 2θ angle using Cu-Kα radiation;
the crystal form V has characteristic peaks at 8.38°±0.20°, 9.15°±0.20°, 13.52°±0.20°, and 18.44±0.20° in an X-ray powder diffraction expressed at a 2θ angle using Cu-Kα radiation.

11. The pharmaceutical composition according to claim 1, wherein the pharmaceutical composition satisfies one or more of the following conditions:
(1) the crystal form III has characteristic peaks at 12.91°±0.20°, 16.77°±0.20°, 19.27°±0.20°, 22.80°±0.20°, 13.75°±0.20°, 14.46°±0.20°, 20.86°±0.20°, 21.08°±0.20°, 23.75°±0.20°, and 24.05°±0.20° in an X-ray powder diffraction expressed at a 2θ angle using Cu-Kα radiation;
(2) the compound of formula A has a Do particle size of 1 to 5 μm;
(3) the compound of formula A has a D₅₀ particle size of 6 to 15 μm;
(4) the compound of formula A has a D₉₀ particle size of 20 to 40 μm;
(5) the compound of formula A has a tap density (bulk density) of 0.32 to 0.5 g/mL.

12. The pharmaceutical preparation according to claim 6, wherein the pharmaceutical preparation is a tablet, a capsule, or a granule.

13. The pharmaceutical preparation according to claim 12, wherein the tablet is a coated tablet, comprising a tablet core and a coating layer; preferably, the tablet core comprises a pharmaceutical composition, which is in a solid form, and comprises an active ingredient and a pharmaceutically acceptable excipient; the active ingredient comprises a compound of formula A: 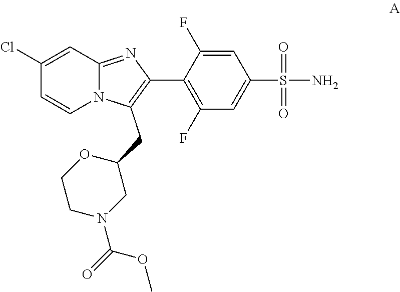 [SMILES: COC(=O)N1CCO[C@@H](Cc2c(-c3c(F)cc(S(N)(=O)=O)cc3F)nc3cc(Cl)ccn23)C1]
the excipient is selected from one, two or more of the following excipients comprising, but not limited to, a diluent, a disintegrant, an adhesive, a glidant, and a lubricant; the compound of formula A is selected from one, two or more of a crystal form I, a crystal form III, and a crystal form V;
the crystal form I has characteristic peaks at 8.56°±0.20°, 12.48°±0.20°, and 22.13°±0.20° in an X-ray powder diffraction expressed at a 2θ angle using Cu-Kα radiation;
the crystal form III has characteristic peaks at 12.91°±0.20°, 16.77°±0.20°, 19.27°±0.20°, and 22.80°±0.20° in an X-ray powder diffraction expressed at a 2θ angle using Cu-Kα radiation;
the crystal form V has characteristic peaks at 8.38°±0.20°, 9.15°±0.20°, 13.52°±0.20°, and 18.44±0.20° in an X-ray powder diffraction expressed at a 2θ angle using Cu-Kα radiation.

14. The pharmaceutical preparation according to claim 13, wherein the pharmaceutical preparation is any one of the following schemes:
scheme 1:
the tablet core of the coated tablet comprises the following components by weight: 10 to 40 parts of the compound of formula A, a total of 50 to 80 parts of lactose monohydrate and microcrystalline cellulose, 0.5 to 6 parts of croscarmellose sodium, 0.5 to 6 parts of hydroxypropyl cellulose, 0.1 to 3 parts of colloidal silica, and 0.1 to 3 parts of magnesium stearate;
the coating layer has a film coating material of Opadry gastric-soluble coating series;
preferably, the compound of formula A is present in the tablet core in the form of a polymorph thereof,
scheme 2:
the tablet core of the coated tablet comprises the following components: 25 mg of the compound of formula A, 49.6 mg of lactose monohydrate, 17.4 mg of microcrystalline cellulose, 3.0 mg of croscarmellose sodium, 3.0 mg of hydroxypropyl cellulose, 1.0 mg of colloidal silica, and 1.0 mg of magnesium stearate;
the compound of formula A is present in the tablet core in crystal form I, crystal form II, crystal form III, crystal form IV, crystal form V, crystal form VI, crystal form VII, crystal form VIII, or crystal form IX;
the coating layer has a film coating material of Opadry gastric-soluble coating series;
scheme 3:
the tablet core of the coated tablet comprises the following components: 100 mg of the compound of formula A, 198.4 mg of lactose monohydrate, 69.6 mg of microcrystalline cellulose, 12.0 mg of croscarmellose sodium, 12.0 mg of hydroxypropyl cellulose, 4.0 mg of colloidal silica, and 4.0 mg of magnesium stearate;
the compound of formula A is present in the tablet core in crystal form I, crystal form II, crystal form III, crystal form IV, crystal form V, crystal form VI, crystal form VII, crystal form VIII, or crystal form IX;
the coating layer has a film coating material of Opadry gastric-soluble coating series.

15. A method for preparing the tablet according to claim 12, comprising compressing a pharmaceutical composition, which is in a solid form, and comprises an active ingredient and a pharmaceutically acceptable excipient; the active ingredient comprises a compound of formula A: 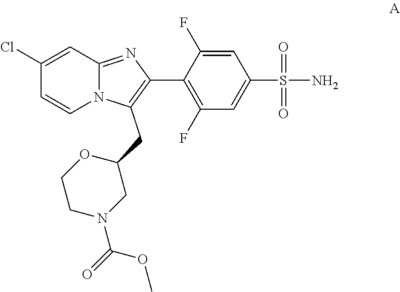 [SMILES: COC(=O)N1CCO[C@@H](Cc2c(-c3c(F)cc(S(N)(=O)=O)cc3F)nc3cc(Cl)ccn23)C1]
the excipient is selected from one, two or more of the following excipients comprising, but not limited to, a diluent, a disintegrant, an adhesive, a glidant, and a lubricant; the compound of formula A is selected from one, two or more of a crystal form I, a crystal form III, and a crystal form V;
the crystal form I has characteristic peaks at 8.56°±0.20°, 12.48°±0.20°, and 22.13°±0.20° in an X-ray powder diffraction expressed at a 2θ angle using Cu-Kα radiation;
the crystal form III has characteristic peaks at 12.91°±0.20°, 16.77°±0.20°, 19.27°±0.20°, and 22.80°±0.20° in an X-ray powder diffraction expressed at a 2θ angle using Cu-Kα radiation;
the crystal form V has characteristic peaks at 8.38°±0.20°, 9.15°±0.20°, 13.52°±0.20°, and 18.44±0.20° in an X-ray powder diffraction expressed at a 2θ angle using Cu-Kα radiation.

16. The method for preparing the tablet according to claim 15, wherein the method for preparing the tablet comprises compressing the pharmaceutical composition by a wet granulation tabletting method, and optionally with or without coating.

17. The method for preparing the tablet according to claim 16, wherein the wet granulation tabletting method comprises: wet granulating the pharmaceutical composition except the lubricant, pelletizing, drying, and pelletizing again to obtain a dry granule; mixing the dry granule with the lubricant, and tabletting;
preferably, in the drying step of the wet granulation tabletting method, the weight loss on drying of the material is controlled at 1.5% to 2.5%;
preferably, in the drying step, when the weight loss on drying of the material is between 1.0% and 2.5%, an inlet air temperature is turned off to stop drying; preferably, in the drying step, an equipment used is a fluidized bed.

18. The method for preparing the tablet according to claim 16, wherein the wet granulation comprises: mixing the compound of formula A, the glidant (e.g., colloidal silica), the diluent (e.g., microcrystalline cellulose and lactose monohydrate), and the disintegrant (e.g., croscarmellose sodium), spraying an adhesive solution (e.g., hydroxypropyl cellulose) into the mixture, and granulating after the adhesive solution is sprayed, optionally with or without water replenishing;
preferably, a method for preparing a coated tablet comprises the following steps:
(1) wet granulating the mixture, pelletizing, drying, and pelletizing again to obtain a dry granule;
(2) mixing the dry granule with the lubricant, and tabletting to obtain a tablet core;
(3) spraying the tablet core with a coating solution to obtain the coated tablet.

19. A method for inhibiting P2X3 in a subject in need thereof, comprising: administering the pharmaceutical composition according to claim 1 to the subject.

20. A method for preventing, curing, treating, or alleviating a disease in animals that is at least partially mediated by P2X3 or related to P2X3 activity or a method for the treatment of pain, pruritus, endometriosis, urinary tract disease, or respiratory disease in a subject in need thereof, comprising: administering the pharmaceutical composition according to claim 1 to the subject.

21. The method according to claim 20, wherein the method satisfies one or more of the following conditions:
(1) the pain comprises: inflammatory pain, surgical pain, visceral pain, dental pain, premenstrual pain, central pain, pain due to burns, migraine, or cluster headache;
(2) the urinary tract disease comprises: urinary incontinence, overactive bladder, dysuria, cystitis, prostatitis, prostatodynia, and benign prostatic hyperplasia;
(3) the respiratory disease comprises: a respiratory disorder, comprising idiopathic pulmonary fibrosis, chronic obstructive pulmonary disease, asthma, bronchospasm, acute cough, or chronic cough; preferably, the pharmaceutical preparation is also capable of reducing the side effects of dysgeusia associated with the treatment.
